(19) United States
(12) Patent Application Publication
(10) Pub. No.: US 2022/0323396 A1
(43) Pub. Date: Oct. 13, 2022

(54) COMPOSITIONS AND METHODS FOR TREATING VIRAL INFECTION USING ANTIVIRAL COCKTAILS

(71) Applicant: LAKHMIR CHAWLA, San Diego, CA (US)

(72) Inventor: LAKHMIR CHAWLA, San Diego, CA (US)

(21) Appl. No.: 17/712,724

(22) Filed: Apr. 4, 2022

Related U.S. Application Data
(63) Continuation of application No. 17/330,031, filed on May 25, 2021.
Application No. 17/330,031 is a continuation-in-part of application No. PCT/US21/34060, filed on May 25, 2021.
(60) Provisional application No. 63/030,011, filed on May 26, 2020.

(51) Int. Cl. A61K 31/245; A61K 38/57; A61K 45/06

(52) U.S. Cl. CPC A61K 31/245; A61K 38/57; A61K 45/06

(57) ABSTRACT

The present disclosure provides compositions and methods of treating a viral infection comprising administering an antiviral cocktail that includes a protease inhibitor and an anticoagulant agent, anti-inflammatory agent and/or an antiviral agent for conjoint administration to a subject.

### RELATED APPLICATIONS

This application is a continuation-in-part of U.S. patent application Ser. No. 17/330,031, filed on May 25, 2021, and International Application No. PCT/US21/34060, filed on May 25, 2021, and claims the benefit of U.S. Provisional Application No. 63/030,011, filed on May 26, 2020. The contents of each of these applications are hereby incorporated by reference herein in its entirety.

### BACKGROUND

The recent and ongoing pandemic of novel Coronavirus (also known as SARS-CoV-2, 2019-nCoV, or COVID-19) is resulting in respiratory viral infections, inflammatory lung injury, and death. While similar to previous Coronavirus epidemics, such as severe acute respiratory syndrome (SARS-CoV) and Middle East Respiratory Syndrome (MERS-CoV), which have higher fatality rates, SARS-CoV-2 appears to be more infectious and, as a result, overall number of deaths from COVID-19 are much greater than that of either SARS or MERS. As of Feb. 21, 2022, SARS-CoV-2 has infected approximately 424 million people with an estimated 5.9 million deaths worldwide. Subjects who develop COVID-19 are provided medical care intended to relieve symptoms and, for severe cases, are provided support for vital organ function.

Currently, information is emerging for COVID-19 patients with physicians observing systemic clotting in the severe and critically ill patients (disseminated intravascular coagulation) with clots showing up in the lungs, kidneys, liver, and heart. D-dimer (a fibrin degradation product indicating thrombosis) is linked to higher odds of death in the hospital. Coronavirus infections are presenting a comorbidity of disseminated intravascular coagulation referred to as COVID-19-associated coagulopathy (CAC), where seriously ill patients are showing elevated fibrinogen and D-dimer levels (3-4 fold) related to coagulation activation from infection/sepsis, cytokine storm and impending organ failure. Clinical professionals are now providing interim guidance for the use of blood thinners (anticoagulants) for potential treatment of the comorbidities associated with COVID-19. As these subjects are meeting the criteria for disseminated intravascular coagulation (DIC), they may be treated with anticoagulants like heparin or nafamostat which is approved for use as an anticoagulant in DIC in Japan.

Methods for effectively managing the infection and the inflammation cytokine response are still to be determined as the relevant information on patient biomarkers and characteristics is developing daily. Improved methods for managing the symptoms of COVID-19 are needed to reduce the demand for ventilators and other equipment, the length of hospitalizations, and the number of deaths resulting from these infections.

### SUMMARY OF THE INVENTION

Protease inhibitors, which are currently used for treating cancer, are also beneficial for the treatment of coronavirus, influenza, Ebola and other serious viral infections. The mode of action of some protease inhibitors relies on serine protease inhibition, which reduces the opportunity for viral entry while alleviating the impact of inflammation and edema associated with viral illness.

One such protease inhibitor is the serine protease inhibitor nafamostat. Appropriate doses of nafamostat to treat all coronaviruses [including, e.g., SARS-CoV-2], influenza, and Ebola virus infections are unknown. This is compounded by the fact that patients with SARS-CoV-2 infection who develop COVID-19 are routinely treated with anticoagulants to prevent clotting (e.g., venous thromboembolism [VTE] prophylaxis). Since these anticoagulants can interact with the anticoagulation effects of nafamostat, the correct dose of nafamostat that is utilized in conjunction with other anticoagulants is critical to understand for both safety and efficacy.

In certain aspects, methods are disclosed herein to treat a viral infection, comprising administering an antiviral cocktail that includes a protease inhibitor at a starting dose of 5-200 mcg/kg/hour over 2 hours. In these methods, the protease inhibitor is administered conjointly with an anticoagulation agent, an anti-inflammatory agent and/or one or more antiviral agent.

In certain aspects, methods are disclosed herein to treat a viral infection, comprising administering an antiviral cocktail that includes a protease inhibitor at a maintenance dose of 10-700 mcg/kg/hour for up to 21 days. In these methods, the protease inhibitor is administered conjointly with an anticoagulation agent, an anti-inflammatory agent and/or one or more antiviral agent.

In certain aspects, methods are disclosed herein to treat hypoxemia, pulmonary intravascular coagulopathy, disseminated intravascular coagulation, viremia, septic shock, cytokine storm, systemic inflammatory response syndrome (SIRS), acute respiratory distress syndrome (ARDS), and/or vascular leak syndrome, comprising administering an antiviral cocktail that includes a protease inhibitor at a starting dose of 5-200 mcg/kg/hour for over 2 hours or a maintenance dose of 10-700 mcg/kg/hour for up to 21 days. In these methods, the protease inhibitor is administered conjointly with an anticoagulation agent, an anti-inflammatory agent and/or one or more antiviral agent.

### DETAILED DESCRIPTION OF THE INVENTION

### General

Disclosed herein are methods of treating a viral infection by administering an antiviral cocktail that includes a protease inhibitor, combined with an anticoagulation agent, an anti-inflammatory agent and/or one or more antiviral agent to a subject with the intent of inhibiting viral propagation and infectivity. Because medically significant viruses such as influenza viruses, coronaviruses and Ebola virus rely on proteolysis of relevant viral proteins for entry, inhibition of the proteases will result in minimizing viral replication and thereby control inflammation, control vascular leak syndrome, and cause disaggregation of platelets and anticoagulation of blood.

The methods disclosed herein determine the type and level of anticoagulant dose (e.g., VTE prophylaxis standard dose with heparin) that may pair with a dose of a protease inhibitor for initiation and maintenance treatment. For example, the methods of the invention include a starting dose and dosing period of the serine protease inhibitor nafamostat (10-100 mcg/kg/hour, over 2 hours) for use in patients with COVID-19, influenza or ebola who are also being treated conjointly with heparin at intermediate anticoagulant dose (>20,000 units per day and aPTT less than 60 seconds). Similarly, the methods of the invention include a maintenance dose and dosing period of nafamostat (1-500 mcg/kg/hour, up to 7 days) in patients with COVID-19, influenza or ebola who are also conjointly treated with heparin at full anticoagulant dose (>20,000 units per day and aPTT greater than 60 seconds).

In addition, disclosed herein are methods of treating hematological and pulmonary conditions with an antiviral cocktail that includes a protease inhibitor, an anticoagulation therapy, an anti-inflammatory therapy and/or one or more antiviral agent. The conditions include hypoxemia, pulmonary intravascular coagulopathy, disseminated intravascular coagulation, viremia, septic shock, cytokine storm, systemic inflammatory response syndrome (SIRS), acute respiratory distress syndrome (ARDS), and/or vascular leak syndrome. The methods may comprise selecting a subject, or a subpopulation of subjects, to determine whom such an antiviral cocktail is suitable for based on selection criteria. For example, vascular leak syndrome is characterized by elevated levels of C-reactive protein and IL-6 biomarkers. Cytokine storm is characterized by increased levels of pro-inflammatory mediators such as IL-1, IL-6 and TNF-α. A subject with elevated levels of such biomarkers may be selected for treatment with the antiviral cocktails of the present invention.

### Definitions

As used in this specification, “a” and “an” can mean one or more. As used herein in the claim(s), when used in conjunction with the word “comprising,” the words “a” and “an” can mean one or more than one. As used herein, “another” can mean at least a second or more.

As used herein, the term “comprising” means that other elements can also be present in addition to the defined elements presented. The use of “comprising” therefore indicates inclusion rather than limitation. The term “consisting of” refers to compositions, methods, and respective components thereof as described herein, which are exclusive of any element not recited in that description of the aspect. As used herein the term “consisting essentially of” refers to those elements required for a given aspect. The term permits the presence of elements that do not materially affect the basic and novel or functional characteristic(s) of that aspect of the disclosure.

Generally, nomenclatures used in connection with, and techniques of, cell and tissue culture, small molecule chemistry, virology, molecular biology, immunology, microbiology, genetics, protein, and nucleic acid chemistry and hybridization described herein are those well-known and commonly used in the art. The methods and techniques of the present disclosure are generally performed according to conventional methods well known in the art and as described in various general and more specific references that are cited and discussed throughout the present specification unless otherwise indicated.

Unless defined otherwise, all technical and scientific terms used herein have the same meaning as commonly understood by one of ordinary skill in the art.

Other than in the operating examples, or where otherwise indicated, all numbers expressing quantities of ingredients or reaction conditions used herein should be understood as modified in all instances by the term “about” whether or not expressly indicated as such. The term “about” when used in connection with percentages, days or dosages or other amounts can mean+/−10%.

As used herein, the terms “administer”, “administering”, and “administered” refer to providing one or several therapeutically active agent(s) to the subject being treated, including “conjoint administration” as defined below. Administration of the protease inhibitor/antiviral, anticoagulation and/or anti-inflammatory agent(s) (i.e., “antiviral cocktail” as defined below) can be carried out on any suitable basis, such as once daily (QD) basis, twice daily (BID), three times daily (TID), four times daily (QID), hourly (“q_h” where “h” denotes the number of hours between doses), or the like, and each day of treatment can be the same or different over the course of treatment. In certain aspects, the antiviral cocktail is administered in the form of one or more liquid solution or suspension introduced to the subject via normal intraperitoneal, subcutaneous, or intravenous delivery techniques.

As used herein, the term “anticoagulating” includes inhibiting or reducing the coagulation of blood. For example, an agent anticoagulates blood if the blood has a longer clotting time in its presence as compared to in its absence.

Accordingly, the phrase “without substantially affecting anticoagulation” refers to a change (or lack thereof) in a blood coagulation parameter such as activated partial thromboplastin time (aPTT), prothrombin time (PT), international normalized ratio (PT/INR), thromboelastography (TEG), or the activated coagulation time (ACT) that is at most 50% (e.g., −50%, −25%, −15%, −10%, −5%, 0%, +5%, +10%, +15%, +25%, +50%). For example, if the systemic ACT of a subject changes from 100 seconds to 110 seconds during a procedure, the procedure does not substantially affect anticoagulation in the subject, since the change in ACT is +10%, which is not more than 50%.

In addition, the phrase “systemic anticoagulation” refers to anticoagulation within a subject's body, which, for example, can be measured from blood drawn directly from the patient. Various coagulation parameter values can be used as a measure of systemic anticoagulation, such as ACT, aPTT, or a combination thereof. In some embodiments, a therapeutically effective change in a coagulation parameter from blood drawn directly from the patient indicates a therapeutically effective systemic anticoagulation.

The term “anti-inflammatory agent”, as used herein, refers to any molecule or compound having an anti-inflammatory effect such as, but not limited to, adrenocortical steroids (cortisol, cortisone, fludrocortisone, prednisone, prednisolone, 6.alpha.-methylprednisolone, triamcinolone, betamethasone, and dexamethasone); non-steroidal anti-inflammatory drugs (NSAIDs) (including salicylates and salicylic acid derivatives, such as aspirin, methyl salicylate, diflunisal, salsalate); para-aminophenol derivatives, i.e., acetominophen) indole and indene acetic acids (indomethacin, sulindac, and etodalac); fenamates; heteroaryl acetic acids (tolmetin, diclofenac, and ketorolac); arylpropionic acids (ibuprofen and derivatives); anthranilic acids (mefenamic acid, and meclofenamic acid); enolic acids (piroxicam, tenoxicam, phenylbutazone, and oxyphenthatrazone); oxyphenbutazone, apazone, ibuprofen, naproxen, naproxen sodium, fenoprofen, ketoprofen, flurbiprofen, piroxicam, diclofenac, etodolac, ketorolac, aceclofenac, nabumetone; nabumetone, gold compounds (e.g., auranofin, aurothioglucose, gold sodium thiomalate); monoclonal antibodies (e.g. tocilizumab) and immunosuppressives (e.g., cyclosporine, tacrolimus (FK-506), sirolimus (rapamycin), azathioprine, or mycophenolate mofetil), and the like.

The term “antiviral agent”, as used herein, refers to therapeutically active agents that can inhibit viral growth. Such agents may include but are not limited to remdesivir (RDV), molnupiravir, nirmatrelvir, ritonavir, galidesivir, ASC09, danoprevir, AT-527, lopinavir, [nirmatrelvir/ritonavir, lopinavir/ritonavir and danoprevir/ritonavir combinations], penciclovir, acyclovir, famciclovir, valacyclovir, tenofovir disoproxil fumarate, lamivudine, zidovudine, didanosine, emtricitabine, stavudine, nevirapine, abacavir, raltegravir, dolutegravir, darunavir, cobicistat, efavirenz, ribavirin, neuraminidase inhibitor, recombinant interferons, recombinant immunoglobulins, oseltamivir, zanamivir, peramivir, baloxavir marboxil, tilorone, favipiravir, [interferons such as IFN-α, IFN-β,1FN-γ, IFN-lambda, peginterferon-α, peginterferon-β and peginterferon-lambda], ribavirin, TAK888, adefovir, amantadine, rintatolimod (Ampligen), amprenavir, umifenovir (Arbidol), atazanavir, brequinar, BLD-2660, SNG001, masitinib, LTX-109, ozanimod, efesovir, novaferon, isoquercetin, ensovibep, plitidepsin, rNAPc2, TTI-0102, TY027, DFV890, rejuveinix, PF-07304814, NA-831, LAU-7b, previfenon, clevudine, ingavirin, silmitasertib, gimsilumab, DWJ1248, IMU-838, elsulfavirine, cenicriviroc, PF-07321332, LY3819253, artesunate, maraviroc, darunavir, cobicistat, PBI-045, PF-07321332, atafenovir, ivermectin, and all pharmaceutically acceptable salts of the above agents. Particularly preferred antiviral agents include remdesivir (RDV), molnupiravir, nirmatrelvir, ritonavir, galidesivir, ASC09, danoprevir, AT-527, lopinavir, and [ASC09/ritonavir, nirmatrelvir/ritonavir, lopinavir/ritonavir and danoprevir/ritonavir combinations]. Therapeutically effective amounts for treatment are familiar to those skilled in the art. Therapeutic activity of a desired agent can be measured using in vitro or in vivo methodology well known to those of skill in the relevant art, for example a desirable therapeutic effect can be assayed in cell culture, assessed in animal testing models and investigated in clinical trials.

As used herein, an “antiviral cocktail” refers to a combination of therapeutically active agents that includes a protease inhibitor together with an anticoagulant agent, anti-inflammation agent and/or an antiviral agent. The cocktail can be provided in the form of a single container or can comprise multiple containers. The antiviral cocktail can also be provided in the form of a kit. In any event, the antiviral cocktail is intended for conjoint administration to a subject in need of antiviral treatment.

As used herein, the term “binding”, sometimes used interchangeably with “interacting”, refers to an association, which may be a stable association, between two molecules due to, for example, electrostatic, hydrophobic, ionic and/or hydrogen-bond interactions under physiological conditions, or the association between an infectious agent such as a virus and a host cell due to, for example, extracellular receptor-ligand interactions under physiological conditions.

A “biomarker” can be anything that can be used as an indicator of a particular physiological state of an organism. For example, a biomarker can be a level of an analyte, metabolite, by-product, mRNA, enzyme, peptide, polypeptide, or protein associated with a particular physiological state, criteria, or score (e.g., the International Society on Thrombosis and Hemostasis (ISTH) DIC criteria, the Japanese Association for Acute Medicine (JAAM) DIC criteria, a clinical evaluation score such as a Sequential Organ Failure Assessment (SOFA) or Acute Physiology and Chronic Health Evaluation (APACHE) score).

In certain embodiments, therapeutically active compounds may be used alone or conjointly administered with another type of therapeutically active agent. As used herein, the phrase “conjoint administration” refers to any form of administration of two or more different therapeutically active agents such that the second agent is administered while the previously administered therapeutically active agent is still effective in the body (e.g., the two agents are simultaneously effective in the subject, which may include synergistic effects of the two agents). For example, the different therapeutically active agents can be administered either in the same formulation or in a separate formulation, either concomitantly or sequentially. In certain embodiments, the different therapeutically active agents can be administered within one hour, 12 hours, 24 hours, 36 hours, 48 hours, 72 hours, or a week of one another. Thus, a subject who receives such treatment can benefit from a combined effect of different therapeutically active agents.

A “cytokine storm” refers to a significant immune typically in response to infection from a pathogen such as a virus and is associated with prolonged inflammation and sepsis in vertebrate tissues. In general terms, a cytokine storm is characterized by an elevation of proinflammatory cytokine levels associated with tissue damage and injury repair due to infection or mechanical stimulus.

The terms “decrease”, “reduce”, “reduced”, “reduction”, “decrease”, and “inhibit” are all used interchangeably herein generally to mean a decrease by a statistically significant amount relative to a reference. However, for avoidance of doubt, such terms typically mean a decrease by at least 10% as compared to a reference level and can include, for example, a decrease by at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, up to and including, for example, the complete absence of the given entity or parameter as compared to the reference level, or any decrease between 10-99% as compared to the absence of a given treatment.

The terms “increased”, “increase” or “enhanced” are all used interchangeably herein generally to mean an increase by a statically significant amount; for the avoidance of any doubt, the terms denote an increase of at least 10% as compared to a reference level, for example an increase of at least about 20%, or at least about 30%, or at least about 40%, or at least about 50%, or at least about 60%, or at least about 70%, or at least about 80%, or at least about 90%, or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or more as compared to a reference level.

The term, “kit” as used herein, means any manufacture (e.g., a package or container) including at least one therapeutically active agent (a protease inhibitor) and instructions for use such as a pharmaceutical label. In certain kits the manufacture may be promoted, distributed, or sold as a single unit or multiple units for performing the methods of the present disclosure.

The term “pharmaceutically acceptable” refers to a material that has been approved or is approvable for pharmaceutical use by a regulatory agency of a relevant federal or state government and/or is listed in the U.S. Pharmacopeia or another generally recognized pharmacopeia for use in animal subjects, and more particularly in humans. A “pharmaceutically acceptable carrier, excipient or vehicle” refers to any vehicle, diluent, adjuvant, excipient or carrier with which a therapeutically active compound is administered.

A “pharmaceutically acceptable salt” refers to a salt of a therapeutically active molecule or compound that is pharmaceutically acceptable and that possesses the desired pharmacological activity of the parent molecule or compound. Pharmaceutically acceptable salts of the therapeutically active agents described herein include those salts derived from pharmaceutically acceptable inorganic and organic acids and bases. Examples of suitable acid salts include acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, formate, fumarate, glucoheptanoate, glycerophosphate, glycolate, hemisulfate, heptanoate, hexanoate, hydrochloride, hydrobromide, hydroiodide, 2-hydroxyethanesulfonate, lactate, maleate, malonate, mesylate (also known as mesylate), methanesulfonate, 2-naphthalenesulfonate, nicotinate, nitrate, oxalate, palmoate, pectinate, persulfate, 3-phenylpropionate, phosphate, picrate, pivalate, propionate, salicylate, succinate, sulfate, tartrate, thiocyanate, tosylate and undecanoate salts. Other acids, such as oxalic, while not in themselves pharmaceutically acceptable, may be employed in the preparation of salts useful as intermediates in obtaining pharmaceutically acceptable acid addition salts. Salts derived from appropriate bases include alkali metal (e.g., sodium and potassium), alkaline earth metal (e.g., magnesium), ammonium and salts.

As used herein, a therapeutic that “prevents” a disorder or condition refers to a compound that, in a statistical sample, reduces the occurrence of the disorder or condition in the treated sample relative to an untreated control sample, or delays the onset or reduces the severity of one or more symptoms of the disorder or condition relative to the untreated control sample.

The term “subject” refers to a mammal, including, but not limited to, a human or non-human mammal, such as a bovine, equine, canine, ovine, or feline selected for treatment or therapy. In some embodiments, the subject is a human. In the specification, the term “patient” is used interchangeably with the term “subject.”

The terms “systemic” or “systemically” as used herein mean, with respect to delivery or administration of a therapeutically active agent to a subject, that such agent is detectable at a biologically significant level in the blood plasma of the subject. The term includes oral or parenteral administration of a therapeutically active agent to a subject.

As used herein, the phrase “therapeutically active” may refer to an activity of a molecule or compound whose effect is consistent with a desirable therapeutic outcome in an intended subject. The phrases “therapeutically active agent”, “therapeutically active protease inhibitor” or a “therapeutically active derivative, variant or modified agent” are used interchangeably herein and refer to a molecule having a therapeutic activity whose effect is consistent with a desirable outcome in a subject and, in the case of a variant, derivative and/or modified molecule, is consistent with the pharmacological activity of the parent molecule. Therapeutic activity may be measured using in vitro or in vivo methodology well known to those of skill in the relevant art, for example a desirable therapeutic effect can be assayed in cell culture, assessed in animal testing models and investigated in clinical trials.

A “therapeutically effective amount” refers to the amount of a therapeutically active agent (molecule or compound) that, when administered to a subject, is sufficient to affect a desired treatment for the infection, disease, condition, complication or disorder present in the subject. The “therapeutically effective amount” of a therapeutically active agent for use in any particular method herein will vary depending on the molecule or compound, the infection, disease, condition, complication or disorder, and its severity and the age and weight of the subject. The full therapeutic effect may not necessarily occur by administration of one single dose of the therapeutically active agent (molecule or compound) and may occur only after administration of a series of doses thereof and/or conjoint administration of multiple therapeutically active agents. A therapeutically effective amount may also vary depending on the identity of the active agent(s), the infection, disease, condition, disorder or complication being addressed (and the severity thereof), as well as the age, weight, adsorption, distribution, metabolism and excretion of the relevant active agent in the subject. Thus, a therapeutically effective amount may need to be administered in one or more administrations to the subject. An appropriate therapeutically effective amount of a therapeutically active molecule or compound can be determined according to any one of several well-established protocols known to those of ordinary skill in the relevant art. For example, animal studies, such as studies using mice, rats or larger mammals, can be used to determine an appropriate dose of a pharmaceutical compound. The results from such animal studies can then be extrapolated to determine doses for use in other species, such as for example, humans.

As used herein, “therapeutically effective rate” includes any rate that leads to an improvement in the treated disease or condition. For example, a rate is therapeutically effective if it leads to curing, relieving, or ameliorating to any extent a symptom of an illness or medical condition or to preventing further worsening of such a symptom.

The term “thrombosis treatment drug” means a substance that inhibits aggregation of blood-clotting proteins and cells related thereto such as platelets.

The term “thrombin” is a serine protease having a central role in hemostasis through the conversion of fibrinogen to fibrin.

The terms “treating” or “treatment” refer to any amelioration, rehabilitation, rejuvenation, improvement, decrease or mitigation of any one or more affect, complication, decrease in normal or preexisting function or capacity, disability or disorder arising from a viral infection in a subject and/or progression or exacerbation of such affect, complication, decrease in normal or preexisting function or capacity, disability or disorder, or of at least one clinical symptom thereof (e.g., stabilization of a discernible symptom), physiologically (e.g., stabilization of a physical parameter), or both, and/or inhibiting at least one physical parameter which may not be discernible to the subject. The terms include prophylactic and/or therapeutic treatments. “Prophylactic” and “therapeutic” treatments are art-recognized and include administration to the host of one or more of the subject antiviral cocktails of the present invention. If administered prior to clinical manifestation of the unwanted condition (e.g., disease or other unwanted state of the host subject) then the treatment is prophylactic (i.e., it protects the host subject against developing the unwanted condition), whereas if it is administered after manifestation of the unwanted condition, the treatment is therapeutic, (i.e., it is intended to diminish, ameliorate, or stabilize the existing unwanted condition or side effects thereof).

### Viral Infections and Antiviral Agents

The methods of the invention are useful for treating viral infection. Viruses are small infectious agents which contain a nucleic acid core and a protein coat, but are not independently living organisms. A virus cannot multiply in the absence of a living cell within which it can replicate. Viruses enter specific living cells either by transfer across a membrane or direct injection, and multiply, causing disease. The multiplied virus can then be released and infect additional cells. Some viruses are DNA-containing viruses and others are RNA-containing viruses. The genomic size, composition, and organization of viruses shows tremendous diversity.

In some aspects of the invention, the viral infection may be caused by an arbovirus, adenovirus, alphavirus, arenaviruses, astrovirus, BK virus, bunyaviruses, calicivirus, cercopithecine herpes virus 1, Colorado tick fever virus, coronavirus, Coxsackie virus, Crimean-Congo hemorrhagic fever virus, cytomegalovirus, Dengue virus, ebola virus, echinovirus, echovirus, enterovirus, Epstein-Barr virus, flavivirus, foot-and-mouth disease virus, hantavirus, hepatitis A, hepatitis B, hepatitis C, herpes simplex virus I, herpes simplex virus II, human herpes virus, human immunodeficiency virus type I (HIV-I), human immunodeficiency virus type II (HIV-II), human papillomavirus, human T-cell leukemia virus type I, human T-cell leukemia virus type II, influenza, Japanese encephalitis, JC virus, Junin virus, lentivirus, Machupo virus, Marburg virus, measles virus, mumps virus, naples virus, norovirus, Norwalk virus, orbiviruses, orthomyxovirus, papillomavirus, papovavirus, parainfluenza virus, paramyxovirus, parvovirus, picornaviridae, poliovirus, polyomavirus, poxvirus, rabies virus, reovirus, respiratory syncytial virus, rhinovirus, rotavirus, rubella virus, sapovirus, smallpox, togaviruses, Toscana virus, varicella zoster virus, West Nile virus, or Yellow Fever virus.

In some aspects, the viral infection may be cause by an enveloped virus. An enveloped virus is an animal virus which possesses a membrane or “envelope,” which is a lipid bilayer containing viral proteins. The envelope proteins of a virus play a pivotal role in its lifecycle. They participate in the assembly of the infectious particle and also play a crucial role in virus entry by binding to a receptor present on the host cell and inducing fusion between the viral envelope and a membrane of the host cell. Enveloped viruses can be either spherical or filamentous (rod-shaped) and include but are not limited to filoviruses, such as Ebola virus or Marburg virus, Arboroviruses such as Togaviruses, flaviviruses (such as hepatitis-C virus), bunyaviruses, and Arenaviruses, Orthomyxoviridae, Paramyxoviridae, poxvirus, herpesvirus, hepadnavirus, Rhabdovirus, Bornavirus, and Arterivirus.

In one aspect of the invention, the viral infection may be caused by Ebola virus. The Ebola virus, which belongs to the filovirus family, is an enveloped virus that has a non-segmented negative-sense RNA genome containing seven genes including glycoprotein (GP). The genomes of the five different ebolaviruses (EDBV, EBOV, RESTV, SUDV and TAFV) differ in sequence and the number and location of gene overlaps. EBOV species are the most dangerous of the known disease-causing ebolaviruses and are responsible for the largest number of outbreaks. EBOV has heterodimeric GP on its surface that consists of GP₁ and GP₂ held together by a disulfide bond. Viral life cycle is initiated with a virion attaching to host cell surface receptors such as C-type lectins, DC-SIGN or integrins with the GP ligand, followed by fusion with the cell membrane and internalization of the resulting endosome. GP₁ and GP₂ are processed by several endosomal proteases including cysteine proteases cathepsin B (CatB) and cathepsin L (CatL). Ebola disease is characterized by outbreaks that typically occur in tropical regions of Sub-Saharan Africa. From the time of the first identification of Ebola virus (1976) through 2013, there were 2,387 confirmed cases with 1,590 overall fatalities. Accordingly, although not widely disseminated, ebolaviruses are extremely deadly, with some outbreaks having up to an 88 to 90% fatality rate. EBOV replicates very efficiently in many host cells, producing large amounts of virus in monocytes, macrophages, dendritic cells and other cells including liver cells, fibroblasts and adrenal gland cells. Viral replication triggers high levels of inflammatory chemical signals and leads to a septic state. The natural reservoir for Ebola virus has not yet been identified; however, bats are considered as the most likely candidate. Ebola virus has also been detected in non-human primate carcasses such as gorillas and chimpanzees. Ebola virus disease is a viral hemorrhagic fever in humans, where symptoms usually begin with fever, sore throat, muscle pain and headaches. Initial symptoms are usually followed by vomiting, diarrhoea, rash and decreased liver and kidney function, after which bleeding, both internal and external, follows.

In another aspect of the invention, the viral infection may be caused by influenza A virus, influenza B virus, and influenza C virus. Influenza type A viruses are divided into subtypes based on two proteins on the surface of the virus. These proteins are called hemagglutinin (HA) and neuraminidase (NA). There are 15 different HA subtypes and 9 different NA subtypes. Subtypes of influenza A virus are named according to their HA and NA surface proteins, and many different combinations of HA and NA proteins are possible. For example, an “H7N2 virus” designates an influenza A subtype that has an HA 7 protein and an NA 2 protein. Similarly, an “H5N1” virus has an HA 5 protein and an NA 1 protein. Only some influenza A subtypes (i.e., H1N1, H2N2, and H3N2) are currently in general circulation among humans. Other subtypes such as H5N1 are found most commonly in other animal species and in a small number of humans, where it is highly pathogenic. Birds are the primary reservoir for Influenza A viruses, especially aquatic birds such as ducks, geese, shorebirds and gulls, but the virus also circulates among mammals including bats, pigs, horses and marine mammals. For example, H7N7 and H3N8 viruses cause illness in horses. Humans can be infected with influenza types A, B, and C. However, the only subtypes of influenza A virus that normally infect people are influenza A subtypes H1N1, H2N2, and H3N2 and recently, H5N1. The influenza A and B viruses that routinely spread in people (human influenza viruses) are responsible for seasonal flu epidemics each year. These seasonal flu infections occur disproportionately in children, however the most severe cases occur in the elderly, the very young and in immunocompromised people. In a typical flu year, influenza viruses can infect up to 5 to 15% of the population, causing up to 3 to 5 million cases of severe illness annually, and accounting for up to 290,000 to 650,000 deaths each year. Flu symptoms range from mild to severe and typically include fever, runny nose, sore throat, muscle pain, headache, coughing and fatigue. However, influenza can progress to pneumonia (viral or secondary bacterial infections), with complications ranging from acute respiratory distress syndrome (ARDS), meningitis, encephalitis and worsening of pre-existing health problems such as asthma and cardiovascular disease. It has recently been reported that there is an association between seasonal flu and venous thromboembolism (VTE).

In yet other aspects of the invention, the viral infection is caused by an arbovirus. Arboviruses are a group of more than 400 enveloped RNA viruses that are transmitted primarily (but not exclusively) by arthropod vectors (mosquitoes, sand-flies, fleas, ticks, lice, etc). Arborviruses have been categorized into four virus families, including the togaviruses, flaviviruses, arenaviruses, and bunyaviruses. Togaviruses includes the genuses Alphavirus (e.g., Sindbis virus, which is characterized by sudden onset of fever, rash, arthralgia or arthritis, lassitude, headache and myalgia) and Rubivirus (e.g., Rubella virus, which causes Rubella in vertebrates). The Flavivirus genus includes yellow fever virus, dengue fever virus, Japanese encaphilitis (JE) virus, and West Nile virus.

Dengue virus is the most common cause of mosquito-borne viral diseases in tropical and subtropical regions around the world and is expanding in geographic range and also in disease severity. Currently, there are no licensed drugs for the treatment of dengue. The virus is a small, enveloped, icosahedral virus, with positive strand RNA of 11,000 nucleotides. There are four distinct serotypes of dengue that cause similar disease symptoms, serotypes 1-4 (DENV-1, DENV-2, DENV-3, and DENV-4) that co-circulate in many areas of the world and give rise to sequential epidemic outbreaks when the number of susceptible individuals in the local population reaches a critical threshold and weather conditions favor reproduction of the mosquito vectors Aedes aegypti and Aedes albopictus. Dengue virus infection causes a characteristic pathology in humans involving dysregulation of the vascular system. In some patients with dengue hemorrhagic fever (DHF), vascular pathology can become severe, resulting in extensive microvascular permeability and plasma leakage into tissues and organs. Recently, the mast cell-derived proteases, tryptase and chymase, have been implicated in the immune mechanism by which dengue induces vascular pathology and shock.

West Nile virus is one of the most widely distributed flaviviruses in the world and has emerged in recent years to become a serious public health threat. West Nile virus is an enveloped positive-strand RNA virus, with a genome that encodes 3 structural and 7 non-structural proteins as a single polypeptide that then co- and post translationally processed to yield the 10 proteins. The 3 virus structural proteins are the capsid (C) protein, pre-membrane protein (prM) which is cleaved during virus maturation to yield the membrane (M) protein and envelope (E) protein. The E protein contains the receptor binding and fusion functions of the virus. Severe viral infection is characterized by fever, convulsions, muscle weakness, vision loss, numbness, paralysis, and coma. Because West Nile virus is capable of eliciting pathology in the brain, it has been postulated that the virus may modulate blood-barrier vascular permeability.

In some alternative aspects of the invention, the viral infection is caused by a respiratory syncytial virus (RSV). Respiratory syncytial virus (RSV) is an enveloped, negative-sense, single-stranded RNA virus of the genus Pneumovirinae and of the family Paramyxoviridae. Symptoms in adults typically resemble a sinus infection or the common cold, although the infection may be asymptomatic. In older adults (e.g., >60 years), RSV infection may progress to bronchiolitis or pneumonia. Symptoms in children are often more severe, including bronchiolitis and pneumonia. The RNA genome of the RSV virus is approximately 15 kb and encodes 11 viral proteins, which includes the F (fusion) protein that is a transmembrane protein of the virus and the M (matrix) protein that is a core protein of the virus. RSV infections are known to cause vascular complications and the infection has been associated with venous thromboembolism.

In other aspects of the invention, the viral infection is caused by a coronavirus. Coronaviruses (CoVs) are a family of enveloped, positive-sense, single-stranded RNA viruses, that was first described in 1949. These viruses are found in mice, rats, bats, dogs, cats, civets, turkeys, horses, pigs, and cattle. Coronaviruses infect humans, and the pathology of these viruses in humans may vary.

The coronavirus genome, approximately 27-32 Kb in length, is the largest found in any of the RNA viruses. Large Spike (S) glycoproteins protrude from the virus particle giving coronaviruses a distinctive corona-like appearance when visualized by electron microscopy. The virus is further classified into 4 groups: the α, β, γ, and δ CoVs by phylogenetic clustering, of which α and β are known to cause infection in humans. It is believed that the gammacoronavirus and deltacoronavirus genera may infect humans. Non-limiting examples of alphacoronaviruses include human coronavirus 229E (229E-CoV), human coronavirus NL63 (NL63-CoV), porcine epidemic diarrhea virus (PEDV), and Transmissible gastroenteritis coronavirus (TGEV). A non-limiting example of a deltacoronaviruses is the Swine Delta Coronavirus (SDCV). Non-limiting examples of betacoronaviruses include Middle East respiratory syndrome coronavirus (MERS-CoV), Severe Acute Respiratory Syndrome coronavirus (SARS-CoV), Severe Acute Respiratory Syndrome coronavirus 2 (SARS-CoV-2), Human coronavirus HKU1 (HKU1-CoV), Human coronavirus OC43 (OC43-CoV), Murine Hepatitis Virus (MHV-CoV), Bat SARS-like coronavirus WIV1 (WIV1-CoV), and Human coronavirus HKU9 (HKU9-CoV).

Coronaviruses facilitate entry to the cell via host protease activation of viral surface proteins (such as TMPRSS2). These host proteases are shown to increase the infectivity of the virus by a thousand-fold.

Viruses may enter cells through the endosomal pathway. For example, SARS-CoV-2 can use the endosomal pathway, which is reliant on the cysteine proteases cathepsin B and L (CatB/L) and it was shown that blocking these proteases prevented infection. Another protein that is relevant to SARS-CoV-2 pathogenesis is angiotensin-converting enzyme 2 (ACE2), which plays a critical role in coronavirus cellular ingress and expressed in the lung and epithelial cells. ACE2 is a type I transmembrane metallocarboxypeptidase which has been investigated by several independent researchers as the coronaviral cellular entry receptor and is also responsible for coronaviral attachment.

The first step of coronavirus entry process is the binding of the N-terminal portion of the viral protein unit S1 to a pocket of the ACE2 receptor. The second step, which is believed to be of utmost importance for viral entry, is the protein cleavage between the S1 and S2 units, which is operated by the receptor transmembrane protease serine 2 (TMPRSS2). The cleavage of the viral protein by TMPRSS2 is a crucial step because, after S1 detachment, the remaining viral S2 unit undergoes a conformational rearrangement which drives and completes the fusion between the viral and cellular membrane, with subsequent entry of the virus into cell, release of its content, replication, and infection of other cells.

Venous thromboembolism has been associated with severe SARS-CoV-2 infection. Because ACE2 receptors limit vasoconstriction, inflammation, and thrombosis in the body, it has been postulated that the entry of SARS-CoV2 into the cells through membrane fusion markedly down-regulates ACE2 receptors, with loss of the catalytic effect of these receptors at the external site of the membrane. Increased pulmonary inflammation and coagulation have been reported as unwanted effects of the down regulation of ACE2 receptor.

Some subjects suffering from coronavirus infection may develop a syndrome known as pulmonary intravascular coagulation wherein immune complexes activate intravascular coagulation that exist primarily in the pulmonary vasculature. Because subjects with some types of viral infections can develop thromboses, these subjects are often treated with varying types and levels of anticoagulation.

In certain aspects of the invention, the methods provided herein comprise treating a viral infection by administering a protease inhibitor optionally conjointly with an antiviral agent. Antiviral agents are pharmaceutical agents that can inhibit viral growth. Such antiviral agents may include, but are not limited to, remdesivir (RDV), molnupiravir, nirmatrelvir, ritonavir, galidesivir, ASC09, danoprevir, AT-527, lopinavir, [nirmatrelvir/ritonavir, lopinavir/ritonavir and danoprevir/ritonavir combinations], penciclovir, acyclovir, famciclovir, valacyclovir, tenofovir disoproxil fumarate, lamivudine, zidovudine, didanosine, emtricitabine, stavudine, nevirapine, abacavir, raltegravir, dolutegravir, darunavir, cobicistat, efavirenz, ribavirin, neuraminidase inhibitor, recombinant interferons, recombinant immunoglobulins, oseltamivir, zanamivir, peramivir, baloxavir marboxil, tilorone, favipiravir, [interferons such as IFN-α, IFN-β,1FN-γ, IFN-lambda, peginterferon-α, peginterferon-β and peginterferon-lambda], ribavirin, TAK888, adefovir, amantadine, rintatolimod (Ampligen), amprenavir, umifenovir (Arbidol), atazanavir, brequinar, BLD-2660, SNG001, masitinib, LTX-109, ozanimod, efesovir, novaferon, isoquercetin, ensovibep, plitidepsin, rNAPc2, TTI-0102, TY027, DFV890, rejuveinix, PF-07304814, NA-831, LAU-7b, previfenon, clevudine, ingavirin, silmitasertib, gimsilumab, DWJ1248, IMU-838, elsulfavirine, cenicriviroc, PF-07321332, LY3819253, artesunate, maraviroc, darunavir, cobicistat, PBI-045, PF-07321332, atafenovir, ivermectin, and all pharmaceutically acceptable salts of the above agents. Particularly preferred antiviral agents include remdesivir (RDV), molnupiravir, nirmatrelvir, ritonavir, galidesivir, ASC09, danoprevir, AT-527, lopinavir, and [ASC09/ritonavir, nirmatrelvir/ritonavir, lopinavir/ritonavir and danoprevir/ritonavir combinations]. Therapeutically effective amounts for treatment are familiar to those skilled in the art.

### Hematological and Pulmonary Conditions

The methods of the invention are useful for treating hematological and pulmonary conditions. Hematological conditions are pathological conditions that primarily affect the blood & blood-forming organs. Pulmonary conditions are conditions that primarily affect the lungs.

In some aspects of the invention, the pulmonary condition is hypoxemia. Hypoxemia refers to the low oxygen levels in the blood in a subject. The condition ultimately reduces oxygen throughout the body. Chronic hypoxemia symptoms include lung tightness, breathlessness, coughing, low lung capacity and volume. Hypoxemia can be caused by injury to the lungs, lung and sinus diseases, lung infections, lung cancer, and a host of medications that can injure lung cells and decrease the production of lung surfactants. Subjects with hypoxemia are usually on oxygen therapy.

Hypoxemia may be defined in terms of reduced partial pressure of oxygen (mm Hg) in arterial blood, but also in terms of reduced content of oxygen (ml oxygen per dl blood) or percentage saturation of hemoglobin (the oxygen binding protein within red blood cells) with oxygen. Acute hypoxemia can cause symptoms such as breathlessness or an increased rate of breathing. However, in a chronic context, and if the lungs are not well ventilated, hypoxemia can result in pulmonary hypertension, overloading the right ventricle of the heart and causing cor pulmonale and right sided heart failure. Severe hypoxemia can lead to respiratory failure. Many subjects with lung or sinus diseases or lung infections experience hypoxemia. The condition may be due to destruction of the alveoli in the lungs or the inadequate production of lung surfactants that enhance oxygen uptake.

In other aspects of the invention, the hematological condition is disseminated intravascular coagulation (DIC). DIC is characterized by a systemic activation of the blood coagulation system, leading to subsequent clot formation, blood vessel obstruction and organ dysfunction. The large consumption of platelets and coagulation factors in this process may in turn cause bleeding, which further worsens the subject's condition and decreases the chances of survival. DIC is usually caused by underlying condition in a subject, such as systemic inflammatory response syndrome (SIRS), sepsis, trauma, malignancy, heat stroke and hyperthermia. SIRS and sepsis are among the most common causes of DIC. Between 30% and 50% of sepsis patients develop DIC and sepsis severity positively correlates with DIC incidence and therefore mortality. Diagnosis of DIC is based on the clinical presentation of the underlying condition, along with abnormalities in laboratory parameters (prothrombin time, partial thromboplastin time, fibrin degradation products, D-dimer, or platelet count). The primary treatment of DIC is to address the underlying condition that is the responsible coagulation trigger. Blood product support in the form of red blood cells, platelets, fresh frozen plasma, and cryoprecipitate may be used to treat or prevent clinical complications.

In still other aspects of the invention, the pulmonary condition is pulmonary intravascular coagulopathy (PIC). Pulmonary intravascular coagulopathy is distinct from disseminated intravascular coagulation. PIC was first coined in McGonagle D, Sharif K, O'Regan A, Bridgewood C Autoimmun Rev. 2020 May 1; 102560 during the COVID-19 pandemic. Key features of COVID-19 related PIC are elevated levels of D-dimers and cardiac enzymes, pulmonary vascular bed thrombosis and fibrinolysis, and emergent pulmonary hypertension induced ventricular stress. Fibrinogen and CRP levels were also both significantly elevated in PIC. PIC is also characterized by hundreds of small blood clots throughout the lungs, which is typically not seen with other types of lung infections. The numerous blood clots are the cause of dramatically decreased blood oxygen levels in severe SARS-COV-2 infection (COVID-19).

In another aspect of the invention, the hematological condition is septic shock. Septicemia is an acute and serious bloodstream infection. Septicemia occurs when a bacterial or viral infection elsewhere in the body, such as in the lungs or skin, enters the bloodstream. Entry of microbesin the blood stream is dangerous because the microbes and their toxins can be carried through the bloodstream to a subject's entire body. Septicemia can quickly become life-threatening and it must be rapidly treated. If it is left untreated, septicemia can progress to sepsis. Sepsis is a serious complication of septicemia. Sepsis is when inflammation throughout the body occurs. This inflammation can cause blood clots and block oxygen from reaching vital organs, resulting in organ failure. When the inflammation occurs with extremely low blood pressure, septic shock occurs. Septic shock is fatal in many cases. Sepsis may manifest into sepsis-induced coagulopathy (SIC) or sepsis-associated coagulopathy (SAC). The International Society on Thrombosis and Hemostasis (ISTH) DIC and the Japanese Association for Acute Medicine (JAAM) DIC provide criteria on determining subject selection for SIC and SAC.

In yet another aspect of the invention, the hematological condition is vascular leak syndrome. Vascular leak syndrome (VLS) is characterized by fever, hypotension, peripheral edema and hypoalbuminemia. VLS can occur as a side effect of illnesses due to pathogens such as viruses and bacteria. VLS is characterized by an increase in vascular permeability accompanied by extravasation of fluids and proteins resulting in interstitial edema and organ failure. Manifestations of VLS include fluid retention, increase in body weight, peripheral edema, pleural and pericardial effusions, ascites, anasarca and, in severe form, signs of pulmonary and cardiovascular failure. Symptoms are highly variable among patients and the causes are poorly understood. Endothelial cell modifications or damage are thought to be important is vascular leak. The pathogenesis of endothelial cell (EC) damage is complex and can involve activation or damage to ECs and leukocytes, release of cytokines and of inflammatory mediators, alteration in cell-cell and cell-matrix adhesion and in cytoskeleton function. Biomarkers to identify vascular leak syndrome include low albumin, elevated C-reactive protein (CRP), and elevated IL-6.

In certain aspects, the methods of the invention are useful for improving cardiac performance in a subject. Improved cardiac performance may be characterized, for example, by decreased pulmonary artery pressure.

In certain other aspects, the methods of the invention are useful for improving pulmonary function in a subject. Improved pulmonary performance may be characterized by decreased respiratory rate, rapid improvement of oxygenation, suppression of fibrinolysis by euglobulin lysis activity, decreased levels of C-reactive protein (CRP), and/or rapid improvement on a 7-point ordinal scale. In certain aspects, the subject is free of respiratory failure. Respiratory failure is defined as the need for mechanical ventilation, extracorporeal membrane oxygenation (ECMO), non-invasive ventilation, or high flow oxygen devices.

### Cytokine Storm

The methods of the invention are useful for inhibiting cytokine storm. Cytokine storm (also known as hypercytokinemia) is a significant immune response to pathogens that invade the body. For example, the influenza A (H1N1) virus may trigger cytokine storms within the body, and COVID-19 infection is also thought to trigger cytokine storm. During a cytokine storm, pro-inflammatory mediators, such as Interleukin-1 (IL-1), Interleukin-6 (IL-6), tumor necrosis factor-alpha (TNF-α), oxygen free radicals, and coagulation factors are released by the immune cells. Accordingly, successful treatment of cytokine storm using the methods of the invention can be assessed by detecting a decrease in plasma concentration of one or more cytokine or chemokine molecule including intracellular adhesion molecule-1 (ICAM-1), vascular cell adhesion molecule-1 (VCAM-1), inducible nitric oxide synthase (iNOS), tumor necrosis factor (TNF-α), interleukin-1α (IL-1α), interleukin-1β (IL-1β), interleukin-2 (IL-2), interleukin-6 (IL-6), interleukin-7 (IL-7), interleukin-10 (IL-10), granulocyte-colony stimulating factor (GCSF), IFN-gamma-inducible protein 10 (IP-10), monocyte chemoattractant protein-1 (MCP1), lipopolysaccharide-induced CXC chemokine (LIX), and macrophage inflammatory protein 1α (MIP1α). Successful treatment can also be characterized by detecting increased expression of anti-inflammatory factors such as Neuregulin (NRG1), Insulin Growth Factor 1 (IGF1) and Hepatocyte Growth Factor (HGF).

Cytokine storms may also be associated with a number of non-infectious diseases, including adult respiratory distress syndrome (ARDS) and systemic inflammatory response syndrome (SIRS). Acute respiratory distress syndrome (ARDS) is a serious lung condition that causes low blood oxygen. Individuals who develop ARDS are usually ill due to another disease or a major injury. In ARDS, fluid builds up inside the tiny air sacs of the lungs, and surfactant breaks down. Surfactant is a foamy substance that keeps the lungs fully expanded so that a person can breathe. These changes prevent the lungs from filling properly with air and moving enough oxygen into the bloodstream and throughout the body. The lung tissue may scar and become stiff.

ARDS develops in response to lung damage due to underlying illnesses such as sepsis, pneumonia, COVID-19 or other issues. ARDS pathogenesis is mediated in part by proteinase-activated receptors (PAR 1-4). PAR 1 and PAR 2 play key roles in mediating the interplay between coagulation and inflammation and tissue repair and fibrosis. PARs are activated by proteases, including serine proteases that are inhibited by serine protease inhibitors such as nafamostat. Nafamostat is a potent inhibitor of PAR-activating proteases such as thrombin and tryptase.

Thrombin activates PAR-1 and PAR-4 in platelets. The downstream effects of PAR-1 include:
Disruption of adherens junctions between cells, increasing lung microvessel permeability
Upregulation of selectins and ICAM (adhesion molecules for neutrophils)
Release of inflammatory mediators:
   IL-1, IL-2, IL-6, IL-8, TNF-α, CCL2
Downstream increase in Tissue Factor (TF) release, which further stimulates coagulation
   Tryptase activates PAR-2 on endothelial cells. Downstream effects of PAR-2 include:
Pro- or anti-inflammatory effects, depending on concentration and local conditions
Release of IL-8
Compromising barrier function and promoting sepsis and acute lung injury in animal models
Suppressing expression of Ve-cadherin
Inducing neutrophil and lung fibroblast migration
Inducing TF expression and von Willebrand factor release, promoting coagulation

Nafamostat inhibits human tryptase activity (e.g., IC₅₀ of 1.6×10⁻¹¹ M). Nafamostat inhibits thrombin activity in a potent, specific and reversible way (e.g., IC₅₀ values ranging from 1.9×10⁻⁶M to 3.3×10⁻⁷).

Non-limiting examples of ARDS biomarkers relevant to nafamostat's mechanism of action include endothelial damage markers, such as Ang-1, Ang-2, ICAM-1, selectins, VEGF, vWF, PA-1, protein C, and coagulation and fibrinolysis markers, such as PA-1, Protein C, thrombomodulin, Tissue Factor, and cell-free hemoglobin. Key PAR-dependent biomarkers in ARDS are listed in Table 1.

TABLE 1
Key PAR-dependent biomarkers in ARDS

| Marker | Function | Diagnostic or Prognostic |
|---|---|---|
| Endothelium damage | | |
| vWF | Secreted multimeric glycoprotein, marker | Prognostic |
| | of endothelial injury: acts as a bridge for | Increased vWIF is associated with |
| | platelet adhesion, and can promote platelet | an increased likelihood of |
| | aggregation | progression to ARDS (debated), |
| | | and decreased survival |
| Selectins | Cell surface lectins that mediate adhesion | Diagnostic and Prognostic |
| | of leukocytes and platelets to endothelial | Increased soluble plasma levels in |
| cells | | ARDS are associated with |
| | | decreased survival |
| ICAM (P- | Soluble intercellular adhesion molecule on | Diagnostic |
| selectin) | endothelia. | Presence is associated with |
| | | increased likelihood of progression |
| | | to ARDS |
| | | Prognostic |
| | | Increased soluble plasma levels in |
| | | ARDS,is associated with decreased |
| | | survival , worse outcomes |
| Coagulation and fibrinolysis | | |
| Tissue | Membrane-bound | activator of Factor Vita, Diagnostic |
| factor | leading eventually to thrombin formation | In ARDS, increased levels of TF in |
| | and fibrin deposition | lung, especially in sepsis-induced |
| | | ARDS |

SIRS is a serious condition related to systemic inflammation, organ dysfunction, and organ failure. It is a subset of cytokine storm, in which there is abnormal regulation of various cytokines. SIRS is also closely related to sepsis and subjects that satisfy criteria for SIRS may also have a suspected or proven infection. SIRS may be generally manifested as a combination of vital sign abnormalities including fever or hypothermia, tachycardia, tachypnea, and leukocytosis or leukopenia. SIRS is nonspecific and can be caused by ischemia, inflammation, trauma, burns, infection, pancreatitis, stress, organ injury, major surgery, fractures, or several insults combined. Thus, SIRS is not always related to infection.

Cytokine storms have the potential to cause significant damage to body tissues and organs. For example, occurrence of cytokine storms in the lungs can cause an accumulation of fluids and immune cells in the lungs and eventually block off the body's airways, thereby resulting in respiratory distress and even death. It has been suggested that pulmonary fibrosis is a potential consequence of severe SARS-CoV-2 infection (COVID-19). During SARS-CoV-2 infection, the immune response in the lungs is robust and, as a result, scar tissue called fibrosis forms. Pulmonary fibrosis is a condition that causes lung scarring and stiffness and impedes proper lung functioning.

### Anticoagulant Therapy

Blood clotting, also known as coagulation, is a process that is essential for the survival of mammals. The process of clotting can be divided into four phases. The first phase, vasoconstriction, decreases blood loss in the damaged area. In the next phase, platelet activation occurs by thrombin formation and the platelets attach to the site of the vessel wall damage forming a platelet aggregate. In the third phase, formation of clotting complexes leads to massive formation of thrombin, which converts soluble fibrinogen to fibrin by cleavage of two small peptides. In the fourth phase, after wound healing, the fibrin clot is dissolved by the action of the key enzyme of the endogenous fibrinolysis system called plasmin.

Two alternative pathways can lead to the formation of a fibrin clot in the coagulation cascade: the intrinsic and the extrinsic pathways. Both pathways comprise a relatively large number of proteins, which are known as clotting factors. The intrinsic and extrinsic pathways are initiated by different mechanisms, but converge to give a common final path of the clotting cascade. In this final path of clotting, clotting factor X is activated (Factor Xa) and is responsible for the formation of thrombin from the inactive precursor prothrombin circulating in the blood.

Venous thromboembolism (VTE) is a condition in which a blood clot forms most often in the deep veins of the leg, groin or arm (known as deep vein thrombosis, DVT) wherein the clot travels through blood circulation, lodging in the lungs (known as pulmonary embolism, PE). Anticoagulants are effective at reducing the risk of blood clots caused by VTE. Anticoagulant therapy prevents blood clots by blocking specific coagulation factors in the coagulation cascade.

In certain aspects, provided herein is a method of treating a viral infection in a subject, comprising administering an antiviral cocktail that includes a protease inhibitor conjointly with anticoagulant therapy. Exemplary anticoagulant therapies are listed in Table 2.

In some aspects of the invention, the anticoagulant therapy is a heparin. Heparins are therapeutically active agents of the glycosaminoglycan family, extracted from natural sources, and have valuable anticoagulant and antithrombotic properties. The molecule has a negative charge density. Heparin is widely used as a clinical anticoagulant for such indications as cardiopulmonary bypass surgery, deep vein thrombosis, pulmonary thromboembolism, arterial thrombosis, and prophylaxis against thrombosis following surgery. Some forms of heparin have average molecular weights from 2 kDa to 30 kDa, such as between 12 kDa and 15 kDa. Heparin functions as an anticoagulant by indirectly inhibiting the enzymatic activity of factor Xa and thrombin through its ability to enhance the action of the plasma anticoagulant protein, antithrombin. Therapeutically effective plasma concentrations of heparin are generally 0.2-0.7 units/ml.

Heparin derivatives used in current clinical anticoagulation therapy include unfractionated heparin (UFH), low molecular weight heparin (LMWH), ultra-low molecular weight heparin (ULMWH) and the synthetic pentasaccharide derivatives fondaparunix and idraparinux. Low molecular weight embodiments of heparin include enoxaparin, dalteparin, fondaparinux, tinzaparin, certoparin, ardeparin, nadroparin, parnaparin, and reviparin and ultra-low molecular weight embodiments of heparin include semuloparin. Low MW heparins act primarily as factor Xa inhibitors, as they enhance antithrombin's anticoagulant effect toward factor Xa to a much greater extent than toward thrombin. Low MW heparins are widely used for longer-term anticoagulant therapy to prevent deep vein thrombosis and have certain advantages over unfractionated heparin. Therapeutically effective plasma concentrations of low MW heparins are generally 0.2-2 units/ml. Low MW heparins have plasma half-lives of 4-13 hours, resulting in prolonged anticoagulation even if the agent is discontinued when bleeding occurs.

In other aspects of the invention, the anticoagulant therapy is argatroban. Argatroban is an oral anticoagulant drug that is approved for patients at risk for thrombosis who cannot be treated with heparin. Therapeutically effective plasma concentrations are about 1 μg/ml. As a derivative of L-arginine, argatroban is a competitive inhibitor of thrombin and only interacts with active site of thrombin. It directly inactivates the activity of thrombin (clotting factor IIa) and has no direct action on the generation of thrombin. The function of argatroban is independent of the anti-thrombin in body. Argatroban inactivates not only thrombin in free state in blood, but also inactivates the thrombin combined with fibrin thrombus, blocks the positive feedback of coagulation cascade, and inhibits the thrombin-induced platelet aggregation even in a very low concentration, which indirectly inhibits the formation of thrombin. Due to a small molecular weight, argatroban can enter the inside of thrombus, directly inactivate the thrombin already combined with fibrin thrombus, and even exhibits an antithrombotic effect against an early-formed thrombosis. Furthermore, argatroban can greatly decrease the level of thrombin-antithrombin complex (TAT) in plasma, effectively reduce the hypercoagulable state of patients, and has very good clinical results in treating chronic thromboembolic disease.

In yet other aspects, the anticoagulant therapy is dabigatran. Dabigatran is a potent, reversible, monovalent direct thrombin inhibitor. Dabigatran reduces the risk of stroke and systemic embolism in patients with non-valve atrial fibrillation. It is also useful in the primary prophylaxis of venous thromboembolic complications in adult patients who underwent surgery for elective total hip arthroplasty or surgery for total knee arthroplasty. Dabigatran inhibits free thrombin, fibrin-linked thrombin, and thrombin-induced platelet aggregation. Dabigatran was first disclosed in International Publication No. WO 1998/37075 (incorporated herein by reference in its entirety), which claims compounds with a thrombin inhibiting and thrombin prolonging action, called 1-methyl-2-[N-[4-(N-n-hexyloxycarbonylamidino) phenyl] aminomethyl] benzimidazol-5-ylcarboxylic acid-N-(2-pyridyl)-N-(2-ethoxycarbonylethyl) amides.

In still other aspects of the invention, the anticoagulant therapy is rivaroxaban. Rivaroxaban is anticoagulant compound 5-chloro-N {[(5S)-2-oxo-3-[4-(3-oxomorpholin-4-yl)phenyl] oxazolidin-5-yl] methyl} (thiophene-2-carboxamide, which was originally disclosed in International Publication No, WO 2001/47919 (incorporated herein by reference in its entirety). Rivaroxaban is a small molecule inhibitor of blood clotting factor Xa and is used in the prevention and treatment of thromboembolic diseases such as myocardial infarction, angina pectoris, reocclusion and restenosis after angioplasty or shunt, stroke, stroke transient ischemic, peripheral arterial obstructive diseases, pulmonary embolism and venous thrombosis.

In other aspects, the anticoagulant therapy is apixaban. Apixaban and its method of manufacture are described in U.S. Pat. Nos. 6,967,208 and 7,396,932, and International Publication Nos WO 2007/001385 and WO2006/13542, the disclosures of which are incorporated herein by reference in their entirety.

In still other aspects, the anticoagulant therapy is edoxaban, Edoxaban is disclosed in International Publication No. WO 20131026553, incorporated herein by reference in its entirety. Edoxaban is a member of the so-called “Xaban-group” and is a low molecular inhibitor of the enzyme factor Xa, which participates in the Hood coagulation system, Therefore, edoxaban is classified as an antithrombotic agent and its possible medical indications are reported to be treatment of thrombosis and thrombosis prophylaxis after orthopaedic operations, such as total hip replacement, as well as for stroke prevention in subjects with atrial fibrillation, the prophylaxis of the acute coronary syndrome and prophylaxis after thrombosis and pulmonary embolism.

In certain aspects of the invention, the anticoagulant therapy is administered at VTE Prophylaxis Dose. For example, the anticoagulant therapy is administered at standard dose. Alternatively, the anticoagulant therapy is administered at intermediate dose, or the anticoagulant therapy is administered at full dose. Dosing amounts of anticoagulant therapies are listed in Table 3. For purposes of disclosure, VTE prophylaxis standard anticoagulant dose, intermediate anticoagulant dose, and full anticoagulant dose are specific to the indicated anticoagulant agents.

TABLE 2
Exemplary Anticoagulant Therapies

| Drug | Chemical Structure |
|---|---|
| Heparin | 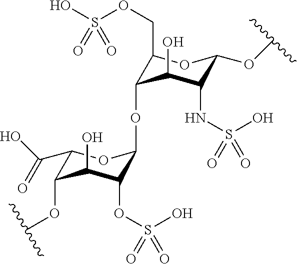 |
| Enoxaparin | 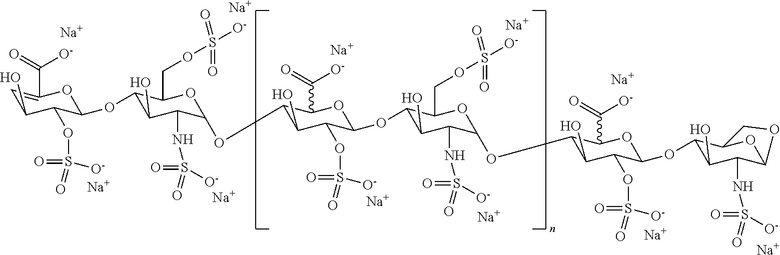 |
| Ardeparin | 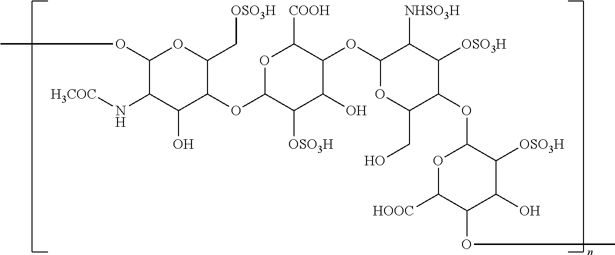 |
| Tinzaparin | 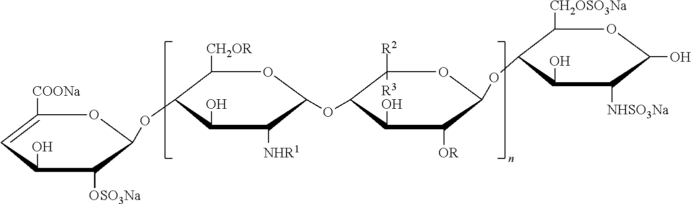 |
| Dalteparin | 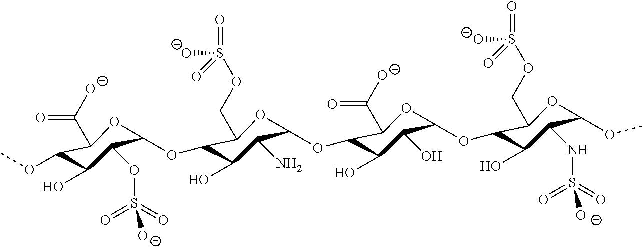 |
| Fondaparinux | 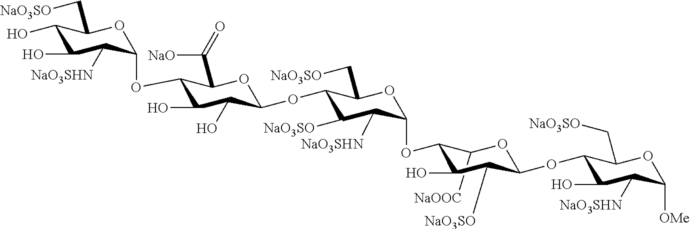 |
| Argatroban | 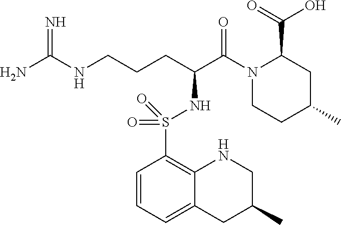 [SMILES: C[C@@H]1CNc2c(cccc2S(=O)(=O)N[C@@H](CCCNC(=N)N)C(=O)N2CC[C@@H](C)C[C@@H]2C(=O)O)C1] |
| Apixaban | 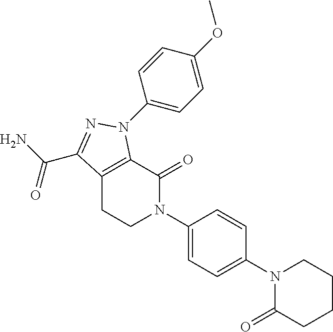 [SMILES: COc1ccc(-n2nc(C(N)=O)c3c2C(=O)N(c2ccc(N4CCCCC4=O)cc2)CC3)cc1] |
| Dabigatran | 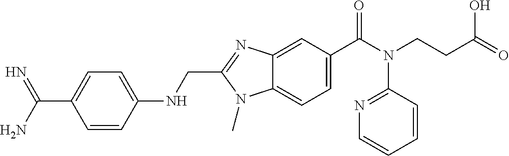 [SMILES: Cn1c(CNc2ccc(C(=N)N)cc2)nc2cc(C(=O)N(CCC(=O)O)c3ccccn3)ccc21] |
| Rivaroxaban | 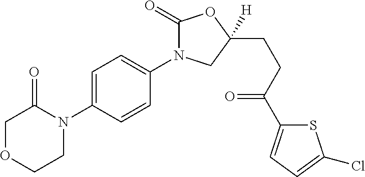 [SMILES: O=C(CC[C@H]1CN(c2ccc(N3CCOCC3=O)cc2)C(=O)O1)c1ccc(Cl)s1] |
| Edoxaban | 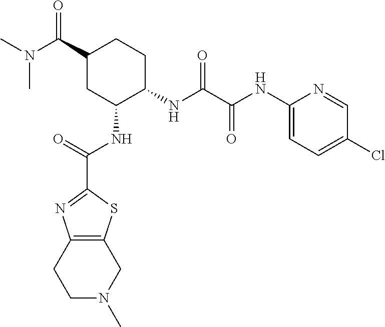 |

TABLE 3
Exemplary Anticoagulant Therapy Doses

| Drug | VTE Prophylaxis | Intermediate | Full Anticoagulant |
|---|---|---|---|
| | Standard Dose | Anticoagulant Dose | Dose |
| Heparin | 10,000-20,000 units/day | Dose >20,000 units | Dose >20,000 units |
| | | per day and aPTT less | per day and aPTT |
| | | than 60 seconds | greater than 60 |
| | | | seconds |
| Enoxaparin | 30-50 mg daily | | 1 mg/kg twice daily |
| Lovenox | | | Or |
| | | | 1.5 mg daily |
| Dalteparin | 2,500−5,000 IU daily | | 200 IU/kg total body |
| | | | weight |
| | | | subcutaneously once |
| | | | daily. |
| Fondaparinux | 2.5 mg daily | | Daily Dose: |
| | | | 5 mg (body weight |
| | | | <50 kg), |
| | | | 7.5 mg (body weight |
| | | | 50 to |
| | | | 100 kg), or |
| | | | 10 mg (body |
| | | | weight >100 kg) |
| Argatroban | N/A | 0.1-0.5 mcg/kg/min | 0.5-1.0 mcg/kg/min |
| Apixabalt | 2.5−5.0 mg daily | | 10-20 mg daily |
| Dabigatran | 200 mg daily | | 300 mg daily |
| | | | (150 mg daily in |
| | | | patients with reduced |
| | | | CrC1) |
| rivaroxaban | 10 mg daily | | 15-20 mg daily |
| Edoxaban | | | 30-60 mg daily |

### Protease Inhibitors

A protease is an enzymatic protein which acts to cleave peptide bonds on proteins. There are several types of proteases, often named for the amino acid target where the cleavage event occurs (serine, threonine, or tyrosine). Certain cleavage events can activate specific cell receptors leading to activation of protein kinases and downstream intracellular signaling pathways. Protein kinases are enzymes which perform the catalyzation of phosphorylation action to amino acids. Downstream effects of kinase activation may include cytokine activation. Cytokines are proteins responsible for cellular signaling and regulatory functions within the body.

Proteases and protein kinases play a vital role in the infection mechanisms for viruses. During some virus infections, like Severe Acute Respiratory Syndrome (SARS) or Dengue, cellular damage triggers kinase activation resulting in inflammation. One such kinase, Protein Kinase R (PKR, a serine/threonine kinase) is activated by proteolytic cleavage and by cytokines type I interferons (IFN-α and -β). This activation in turn can result in apoptosis of the cell through action by eukaryotic translation initiation factor 2 (eIF2α), which may be important in virus replication since it is responsible for regulating mRNA translation.

Other kinases may be activated by coronavirus proteolytic events, which have the ability to facilitate autophosphorylation eIF2α such as PKR-like endoplasmic reticulum kinase (PERK) and general control nonderepressible-2 kinase (GCN2). There are also avenues where PKR activation may lead to apoptosis without activation of eIF2α, and there is evidence that kinases other than PKR are involved in the eIF2a phosphorylation, which is hypothesized to facilitate the coronavirus infection cycle. For influenza viral infection, important signaling pathways are nuclear factor (NF-κB) signaling, PI3K/Akt pathway, MAPK pathway, PKC/PKR signaling, and TLR/RIG-I signaling cascades, all of which are facilitated by protein kinase activities that are activated by proteases. The kinase activation that occurs with these virus infections can result in organ damage from the resulting inflammation and coagulation.

Protease inhibitors can interfere with the mechanisms of cell signaling through prevention of kinase activation. The medical and scientific community understand the opportunity for management of diseases through modulation of protein signaling through kinases and a growing body of knowledge indicates that protease inhibitors may provide specific utility in the management of virus infections such as coronavirus, influenza virus, Ebola virus, Dengue, etc., and may provide some relief to the inflammation symptomology of virus infections. Currently, there are several protease inhibitors (PI) that have been used or investigated clinically to treat a variety of patient populations including those with pancreatitis, chronic obstructive pulmonary disease (COPD), cancer, arthritis, hypertension, and those in need of anticoagulation. In addition, some proteases are an effective treatment option for treating subjects with viral infections.

Often, viral infection relies on the proteolytic activation of host proteins as part of the cellular ingress mechanism. The human protease TMPRSS2 is the primary target for the protease inhibitor component of the antiviral cocktail compositions of the invention due to its importance in SARS-CoV-2 virus infection and pathogenesis. Coronavirus is not the only agent which may be subject to treatment with protease inhibitors. Other viruses like Ebola virus, West Nile virus and Dengue virus and coronaviruses have been investigated for treatment using nafamostat and other protease inhibitors which help prevent infection by such viruses, but also show an alteration of the inflammatory cytokine response.

The reduction of cytokine response may be clinically important as many virally infected subjects suffer immunomodulated impacts of the virus infection through the “kinase cascade” resulting in enhanced morbidity and mortality with some viruses. Protein kinases are responsible for signaling pathways regulating inflammation which may be exacerbated by viral triggered, pro-inflammatory cytokine storms which damage organ tissue.

In certain aspects of the present invention, provided herein are methods for treating a viral infection comprising administering an antiviral cocktail that includes a protease inhibitor and an anticoagulation therapy, an anti-inflammatory therapy and/or one or more antiviral agent. In some aspects, the protease inhibitor is administered conjointly with an anticoagulation therapy. In other aspects, the protease inhibitor is administered at a starting dose of 5 mcg/kg/hour, 10 mcg/kg/hour, 25 mcg/kg/hour, 50 mcg/kg/hour, 75 mcg/kg/hour, 100 mcg/kg/hour, 125 mcg/kg/hour, 150 mcg/kg/hour, 175 mcg/kg/hour, or 200 mcg/kg/hour. In alternative aspects, the protease inhibitor is administered at a maintenance dose 10 mcg/kg/hour, 50 mcg/kg/hour, 100 mcg/kg/hour, 150 mcg/kg/hour, 200 mcg/kg/hour, 250 mcg/kg/hour, 300 mcg/kg/hour, 350 mcg/kg/hour, 400 mcg/kg/hour, 450 mcg/kg/hour, 500 mcg/kg/hour, 550 mcg/kg/hour, 600 mcg/kg/hour, 650 mcg/kg/hour, or 700 mcg/kg/hour.

In certain aspects, the protease inhibitor is administered at a starting dose for over 2 hours, 3 hours, 4 hours, 5 hours, 6 hours, 7 hours, 8 hours, or 9 hours.

In other aspects, the protease inhibitor is administered at a maintenance dose for up to 7 days, 9 days, 11 days, 13 days, 15 days, 17 days, 19 days, or 21 days.

Administration of the antiviral cocktail that includes a protease inhibitor reduces viremia in a subject. In some aspects of the invention, administration of the antiviral cocktail that includes a protease inhibitor results in clearance of the viral infection. Viral clearance may be determined by resolution of viral infection symptoms, or by determining reduction or elimination of viral load in the infected subject using diagnostic techniques well known to those of ordinary skill in the art.

In certain aspects, the protease inhibitor is a serine protease inhibitor. Exemplary serine protease inhibitors are listed in Table 3.

TABLE 3
Exemplary Serine Protease Inhibitors

| Serine Protease | |
|---|---|
| Inhibitor | Chemical Structure |
| Camostat mesylate | 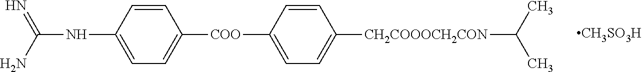 |
| Nafamostat mesylate | 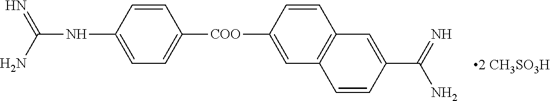 |
| Gabexate mesylate | 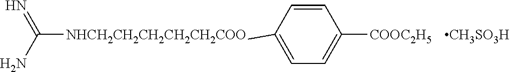 |

In some aspects of the invention, the protease inhibitor (the protease inhibitor therapeutically active agent) used in the practice of the disclosed methods herein is nafamostat, nafamostat mesylate, or another pharmaceutically acceptable salt form of nafamostat. For any protease inhibitor (such as nafamostat) used in the practice of the invention, the selected salt form can be any pharmaceutically acceptable salt form. Nafamostat is a small molecule, broad spectrum, serine protease inhibitor that inhibits thrombin at the platelet thrombin receptor PAR1.

Nafamostat is efficacious due to its inhibition of a broad spectrum of serine proteases involved in a variety of signaling pathways involved in inflammation and reverse vascular leakage which are also involved in facilitating virus infection and propagation.

Nafamostat's function as a therapeutically effective anticoagulation agent has been shown to have positive outcomes for those subjects that have received personalized heparinization during treatment for disseminated intravascular coagulation (DIC).

Nafamostat inhibits virus proteins targets like Coronavirus S-Protein, and human cellular proteins involved in virus infection pathways, particularly serine protease TMPRSS2, which is critical for viral spread and pathogenesis in an infected host. Additional inhibited targets are NF-κB and endosomal protein cathepsin B.

Nafamostat inhibits pro-inflammatory cytokines typically associated with a cytokine storm such as interleukin-1 (IL-1) and interleukin-8 (IL-6). Nafamostat also inhibits proteases within the VIIa complex. Nafamostat inhibits thrombin production, as well as the proteases human Hageman factor, prothrombin, trypsin-1, and kallikrein-1. Nafamostat has been shown to provide broad spectrum inhibition to the coagulation-fibrinolysis system (thrombin, XIIa, Xa, VIIa, and plasmin), the kallikrein-kinin system (kallikrein), the complement system (Clr, Cls, B, D) and pancreatic enzymes (trypsin, pancreatic kallikrein).

Nafamostat has a strong inhibitory action on the coagulation-fibrinolysis system (XIIa, Xa, VIIa, and plasmin), the kallikrein-kinin system (kallikrein), the complement system (Clr, Cls, B, D) and pancreatic enzymes (trypsin, pancreatic kallikrein) that can be demonstrated using in vitro and/or in vivo methods well known to those of ordinary skill and experience in the art.

Platelet activation and aggregation occurs when a subject enters a proinflammatory state. The unique ability of nafamostat to inhibit platelet aggregation and disaggregate platelets in normal human platelet rich plasma provides an important basis to support the clinical use of nafamostat in prolonging the life of a virally infected subject with complications such as platelet thrombosis.

Particularly preferred antiviral cocktails include nafamostat combined with an antiviral agent selected from interferons (such as IFN-α, IFN-β,1FN-γ and IFN-lambda, or pegylated versions thereof) remdesivir (RDV), molnupiravir, nirmatrelvir, ritonavir, galidesivir, ASC09, danoprevir, AT-527, lopinavir, and [ASC09/ritonavir, nirmatrelvir/ritonavir, lopinavir/ritonavir and danoprevir/ritonavir combinations]. For example, nafamostat combined with an interferon or a pegylated interferon, nafamostat combined with remdesivir, nafamostat combined with molnupiravir, nafamostat combined with the nirmatrelvir/ritonavir combination, nafamostat combined with galidesivir, nafamostat combined with the ASC09/ritonavir combination, nafamostat combined with the danoprevir/ritonavir combination, and nafamostat combined with AT-527.

Manufacture of a pharmaceutical composition that contains one or more of the therapeutically active agents of the antiviral cocktails of the present invention (the protease inhibitor, anticoagulant, anti-inflammatory and/or antiviral agents) dissolved or dispersed therein is well understood in the art and generally need not be limited based on formulation. Some therapeutically active agents, such as antivirals, can be provided as a tablet or capsule for oral administration. However, typically such compositions are prepared as an injectable either as liquid solutions or suspensions; however, solid forms suitable for solution or suspension in liquid prior to use can also be prepared. The preparation can also be emulsified or presented as a liposome composition. The one or more therapeutically active agents can be mixed with excipients which are pharmaceutically acceptable and compatible with the selected agent(s) and in amounts suitable for use in the methods described herein. Suitable excipients are, for example, water, saline, dextrose, glycerol, ethanol or the like and combinations thereof. In addition, if desired, a pharmaceutical composition can contain minor amounts of auxiliary substances such as wetting or emulsifying agents, pH buffering agents, and the like which enhance the effectiveness of the active agent(s). The composition of the present disclosure can include pharmaceutically acceptable salts of the components therein. Pharmaceutically acceptable salts include the acid addition salts (formed with the free amino groups of the polypeptide) that are formed with inorganic acids such as, for example, hydrochloric or phosphoric acids, or such organic acids as acetic, tartaric, mandelic, and the like. Salts formed with the free carboxyl groups can also be derived from inorganic bases such as, for example, sodium, potassium, ammonium, calcium or ferric hydroxides, and such organic bases as isopropylamine, trimethylamine, 2-ethylamino ethanol, histidine, procaine, and the like. Pharmaceutically acceptable carriers, excipients and vehicles are well known in the art. Exemplary liquid carriers are sterile aqueous solutions that contain no materials in addition to the active agents and water, and/or can contain a buffer such as sodium phosphate at physiological pH value, physiological saline or both, such as phosphate-buffered saline. In certain aspects of the disclosure, aqueous carriers can contain more than one buffer salt, as well as salts such as sodium and potassium chlorides, dextrose, polyethylene glycol and other solutes. Liquid compositions can also contain liquid phases in addition to and to the exclusion of water. Exemplary of such additional liquid phases are glycerin, vegetable oils such as cottonseed oil, and water-oil emulsions. The amount of the one or more therapeutically active agent(s) used in the present methods that will be effective in the treatment of viral infection in a subject will depend on the nature of such pathogen and can be determined by standard clinical techniques.

The decision to combine one or more of the therapeutically active agents of the present antiviral cocktails into one or more container is based upon simple considerations such as solubility/insolubility in selected carriers and excipients, buffering needs, relative chemical stability profiles, and potential inter-molecule interactions between the selected agents. Accordingly, the antiviral cocktails of the present invention can be provided in from 1 to 4 containers for conjoint administration to a subject in the practice of the current methods.

Each therapeutically active agent component of the present antiviral cocktails is included in its relevant composition in an amount sufficient to exert a therapeutically useful effect in the absence or minimization of undesirable side effects in the subject. Such therapeutically effective concentration may be predicted empirically by testing the agents in in vitro and in vivo systems well known to those of skill in the art and then extrapolated therefrom for dosages for humans. Human doses are then typically fine-tuned in clinical trials and titrated to bring about the desired therapeutic response. To formulate a composition, the weight fraction of a therapeutically active agent is dissolved, suspended, dispersed or otherwise mixed in a selected carrier, excipient or vehicle at an effective concentration. The formulated pharmaceutical compositions containing the one or more active agent(s) can then be conventionally administered in the form of a unit dose, for example. The term “unit dose” when used in reference to a pharmaceutical composition refers to physically discrete units suitable as unitary dosage for the subject, each unit containing a predetermined quantity of the one or more therapeutically active agent(s) calculated to produce the desired pharmacological effect in association with a pharmaceutically acceptable carrier, excipient or vehicle. Examples of unit dose forms include ampoules and pre-filled syringes. Thus, in one preferred aspect of the disclosure, the antiviral cocktail is provided in the form of one or more pharmaceutical composition that includes water for injection. In related aspects, a syringe comprising a therapeutically effective amount of one or more of the therapeutically active agents in a pharmaceutical composition is provided. Unit-dose forms may be administered in fractions or multiples thereof. A multiple dose form is a plurality of identical unit dose forms packaged in a single container to be administered in segregated unit dose form. Examples of multiple dose forms include vials, bottles of tablets or capsules or bottles of pints or gallons. Hence, a multiple dose form is a multiple of unit doses which are not segregated in packaging. Alternatively, the antiviral cocktail is provided in a kit (e.g., a package or container) including at least one therapeutically active agent component of the cocktail. In certain kits the manufacture may be labeled, promoted, distributed, or sold as a unit for performing the methods of the present disclosure.

As discussed herein above, preferred routes of administration for the present compositions are oral (if possible), but most typically parenteral, e.g., via intravenous, intramuscular, intraperitoneal, intradermal or subcutaneous injection. Solutions or suspensions used for such parenteral application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates and agents for the adjustment of tonicity such as sodium chloride or dextrose. The pH of a composition can be adjusted with acids or bases, such as hydrochloric acid or sodium hydroxide. Parenteral preparations can be enclosed in ampoules, disposable syringes or multiple dose vials made of glass or plastic. Pharmaceutical compositions suitable for injection include sterile aqueous solutions (where water soluble) or dispersions, emulsions or suspensions and sterile powders for the extemporaneous preparation of sterile injectable solutions or dispersions. For intravenous administration, suitable carriers comprise physiological saline, bacteriostatic water, Cremophor EL.TM. (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier or vehicle can be a solvent or dispersion medium containing, for example, water, ethanol, polyol (for example, glycerol, propylene glycol, and liquid polyethylene glycol, and the like), and suitable mixtures thereof. The proper fluidity of a composition can be maintained, for example, by the use of a coating such as lecithin, by the maintenance of the selected particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In some cases, isotonic agents are included in the composition, for example, sugars, polyalcohols such as mannitol, sorbitol, or sodium chloride. Prolonged absorption of an injectable composition can be achieved by including in the composition an excipient that delays absorption, for example, aluminum monostearate or gelatin.

In one aspect of the invention, sterile injectable solutions can be prepared by incorporating the selected therapeutically active agent(s) in a specified amount in an appropriate solvent with one or a combination of ingredients enumerated above, as needed, followed by filtered sterilization. Generally, dispersions are prepared by incorporating the selected agent(s) into a sterile vehicle that contains a basic dispersion medium and other ingredients selected from those enumerated above or others known in the art. In the case of sterile powders for the preparation of sterile injectable solutions, the methods of preparation typically include vacuum drying and freeze-drying which yields a powder of the active agent(s) plus any additional desired ingredient from a previously sterile-filtered solution thereof

### Methods of Delivering an Antiviral Cocktail Including a Protease Inhibitor Agent to a Subject

Contemporary methods exist to deliver protease inhibitors to a subject including by any suitable parenteral route including, but not limited to, intravenous, intramuscular, subcutaneous, inhalation, nasal, mucosal, sublingual, and other known routes, as well as standard oral routes of administration. In the case of suspected viral infection and in advance of the onset of symptoms, a subject suspected of viral infection can be provided a single oral dosage form containing a therapeutically effective amount of an antiviral cocktail composition of the present invention to inhibit inflammation onset and potentially interfere with the virus infection pathway. For onset of symptoms, but before severe inflammation, one or more component of the antiviral cocktail can be delivered via inhalation/intranasal delivery for direct delivery of the therapy to the sites of infection. For advanced conditions, where lung function is reduced, and inflammation is severe, the subject could be infused as a part of the standard of care through intravenous application of the antiviral cocktail to the bloodstream.

Parenteral administration of the antiviral cocktails of the present invention is typically carried out intravenously by way of a catheter such as a central venous catheter line or like IV catheter. Alternatively, the cocktail composition(s) can be administered via intravenous, intramuscular, intraperitoneal or subcutaneous injection using a standard needle and syringe. In certain aspects, the cocktail composition(s) can thus be simply formulated to include a suitable injection vehicle such as water for injection. In yet other aspects, the cocktail composition(s) can be administered using an external drug pump such as an infusion pump. In the practice of the methods of the present disclosure, the therapeutically active agent components of cocktail can be present in the composition(s) in the form of a solution, suspension or emulsion.

In certain aspects, the conjoint administration dosing regimen entails classical titration of one or more of the therapeutically active agent components in either ascending or descending doses, for example wherein the first administration is carried out at an initial dose of at least the minimal effective dose of the agent on day 1 of the treatment period and finishes at a second, higher dose, with any number of different or same intervening doses carried out between such first and second doses. Alternatively, titration of an agent can entail an initial (day one) high dose of the agent and ending with a final dose of at least the minimal effective dose of the agent, again with any number of different or same intervening doses carried out between such initial and final doses. In any titration strategy, it may be preferred to administer the therapeutically active agent component at a first high dose approaching the median toxic dose (MTD) for that molecule, or at least approaching the maximum dose of the therapeutic window for the administered agent, followed by a subsequent dose (or doses) at lower level.

In other aspects of the disclosure, the conjoint administration regimens can be carried out multiple times (e.g., repeated), with a so-called “drug holiday”, that is, by following a structured treatment interruption, tolerance break or treatment break, e.g., where subsequent treatment(s) occur from 2 to 7 days after completion of the initial treatment. Here again, for any particular subject, specific conjoint administration dosing regimens can be adjusted over time according to the individual need and the professional judgment of the person administering or supervising the administration of the present antiviral cocktails and the dosing strategies set forth herein are exemplary only and are not intended to limit the scope or practice of the claimed methods. In one particular regimen, the first or first few administrations of a component of the antiviral cocktail are carried out in an intensive care setting where the subject has a catheter such as a central venous catheter line or like IV catheter. Upon stabilization and move to a step-down unit, recovery unit, or other suitable setting, subsequent administrations of the antiviral cocktail can then be carried out using a standard needle and syringe. Subsequent treatment regimens (for example after a drug holiday) can be carried out using an implant or an external drug pump. Subsequent treatment regimens can target the same high dose, short duration of administration period as the initial treatment, or can target lower dose administration of the antiviral cocktail component with or without an extended duration of treatment.

In some aspects of the invention, before delivering the antiviral cocktail to a subject, the subject (or a subpopulation of subjects) can be selected as described in the next section.

### Methods of Selecting Subjects for Delivery of an Antiviral Cocktail Including a Protease Inhibitor Agent

In some aspects, the disclosure relates to methods of selecting a subject, or a subpopulation of subjects, to whom the protease inhibitor component and/or the anticoagulant component of the antiviral cocktail will be delivered from an extracorporeal circuit. These methods include determining that a subject meets certain criteria and then selecting the subject for delivering the therapeutically active agent component(s) from an extracorporeal circuit to the subject. The set of criteria that can be used include the following: (1) criteria for sepsis-induced coagulopathy; (2) criteria for sepsis-associated coagulopathy; (3) criteria of the ISTH for DIC; (4) criteria of the JAAM for DIC; (5) having a plasma procalcitonin level above its healthy reference range; (6) having a plasma nucleosome level above its healthy reference range; and (7) having a plasma syndecan-1 level and/or D-dimer level above its healthy reference range. In some aspects of the invention, the criteria include those enumerated as (1) through (4). In some other aspects, the criteria also include at least one, at least two, or all three enumerated as (5) through (7).

In certain aspects, the method of selecting subjects relates to selecting a subpopulation of subjects that have DIC. Diagnosing DIC facilitates sepsis management and is associated with improved outcomes. Although the ISTH has proposed criteria for diagnosing overt DIC, these criteria are not suitable for early detection. Moreover, no single biomarker can effectively diagnose DIC in subjects with sepsis. The methods disclosed herein address these problems by identifying subjects that will ultimately benefit from treatment with the antiviral cocktails of the present invention.

In certain aspects, the method of selecting subjects relates to selecting a subpopulation of subjects that have PIC. Diagnosing PIC facilitates management of infection management and intravascular thromboses. The hallmarks of PIC are increased D-dimer in the background of an infection. Subjects with PIC typically have normal fibrinogen levels with elevated D-dimer. They may show signs and symptoms of macrophage activation syndrome (MAS) with features of hemophagocytosis, elevated hepcidin levels, and hypoferremia.

A biomarker that can be used for subject selection is procalcitonin (PCT). PCT levels can be significantly elevated in subjects with sepsis and DIC compared to healthy controls. Interestingly, PCT levels can also be significantly higher in subjects with DIC as compared to subjects with sepsis without substantial coagulopathy. Thus, plasma PCT levels can be a useful prognostic marker in detecting not only sepsis but also the progression of onset to DIC. Additionally, PCT may have a potential role in early risk stratification and prediction of overall morbidity and mortality.

Another biomarker that can be used for subject selection is D-dimer. D-dimer is a fibrin degradation product that is present in the blood after a blood clot is degraded by fibrinolysis. It contains two D fragments of the fibrin protein joined by a cross-link. D-dimer concentration may be determined in a subject by a blood test. Reference ranges for D-dimer in non-pregnant adults is less than or equal to 287 ng/mL. D-dimer is associated with the fragmentation of fibrin in coagulopathy and is currently used to identify pulmonary embolisms.

Another biomarker that can be used for subject selection is Interleukin-6 (IL-6). IL-6 is a major pro-inflammatory cytokine and consists of 212 amino acids with two N-linked glycosylation sites. IL-6 signaling is mediated by the binding of IL-6 to either soluble or surface bound IL-6 receptor chain (IL-6R), enabling interaction of the complex with the cell surface transmembrane gp130 subunit. The interaction mediates intracellular signaling and is responsible for the proliferation and differentiation of immune cells. IL-6 plays a crucial role in coagulation; it is primarily involved in the up-regulation of tissue factors that initiate of coagulation. IL-6 is also one of the major cytokines that is released from the lung in response to a wide variety of inflammatory stimuli during pulmonary intravascular coagulation. IL-6 can be measured in serum of the subject using methods well known in the art.

Another biomarker that can be used for subject selection is C-reactive protein (CRP). CRP is a pentraxin family member and is secreted by the liver. CRP may increase in response to either acute or chronic inflammation. CRP levels increase in response to macrophage and adipocyte secretion of IL-6 and lead to activation of the complement pathway. CRP levels may increase in response to microbial infection, inflammation, and tissue damage. As an acute-phase protein, levels of CRP can rise rapidly upon inflammation and thus CRP can function as a biomarker of active inflammation. Moreover, CRP can have a relatively short half-life and thus can also be used to monitor resolution of the inflammatory insult. CRP can be measured in the blood using a high-sensitivity C-reactive protein (hs-CRP) test.

Another biomarker that can be used for subject selection is a nucleosome level. Nucleosome levels can be significantly elevated in subjects with overt DIC compared to healthy controls and compared to septic subjects without DIC. This specific elevation of nucleosomes in subjects with severe coagulopathy suggests that nucleosome level may be useful as a tool to identify subjects with sepsis having overt DIC from subjects with sepsis without coagulopathy.

Another biomarker that can be used for subject selection is syndecan-1. One of the pathophysiological processes in sepsis is endothelial dysfunction, which leads to DIC. Syndecan-1 is a major structural component of the endothelium and plays a key role in endothelial function. Syndecan-1 levels can be associated with not only the severity of illness and mortality but also DIC development in sepsis, thus syndecan-1 can be used as a predictive marker of DIC. In subjects with sepsis, syndecan-1 can correlate with the DIC score and can have strong discriminative power for the prediction of DIC development. Thus, syndecan-1 can be used as a predictive marker of DIC in subjects with sepsis.

Another biomarker that can be used for subject selection is von Willebrand factor (vWF). vWF is a secreted multimeric glycoprotein that acts as a bridge for platelet adhesion, and can promote platelet aggregation. It is a marker of endothelial injury, and in ARDS, increased vWF can be associated with an increased likelihood of progression to ARDS, and decreased survival. Thus, vWF can be used as a predictive marker of ARDS progression in subjects with sepsis, pneumonia, COVID-19, or other conditions that lead to ARDS, targeting those subjects for early intervention with the antiviral cocktail treatment methods of the present invention.

Another set of biomarkers that can be used for subject selection are selectins. Selectins are cell surface lectins (ICAM, a P-selectin, is one example) that mediate adhesion of leukocytes and platelets to endothelial cells, and increased soluble plasma levels of selectins in ARDS are associated with increased likelihood of progression to ARDS and decreased survival. Thus, detection of soluble levels of selectins can be used as a predictive marker of ARDS progression in subjects with sepsis, pneumonia, COVID-19, or other conditions that lead to ARDS, targeting those subjects for early intervention with the antiviral cocktail treatment methods of the present invention.

Another biomarker that can be used for subject selection is Tissue factor (TF). TF is a membrane-bound activator of Factor VIIa, leading eventually to thrombin formation and fibrin deposition in the fibroproliferative phase of ARDS. Increased levels of TF indicate ARDS, especially in sepsis-induced ARDS. TF can be used as a predictive marker of ARDS progression in subjects with sepsis, pneumonia, COVID-19, or other conditions that lead to ARDS, targeting those subjects for early intervention with the antiviral cocktail treatment methods of the present invention.

Beyond reliance on biomarkers, other criteria can also be used to classify subjects. Two such different criteria for evaluating coagulopathy in sepsis include the following: sepsis-induced coagulopathy (SIC) and sepsis-associated coagulopathy (SAC). Both detection techniques use universal hemostatic markers of platelet count and pro-thrombin time. Additional criteria include the following: ISTH DIC criteria and the JAAM DIC criteria.

Coagulation factors and anticoagulant proteins not only play a role in hemostatic activation, but also interact with specific cell receptors leading to activation of signaling pathways. Specifically, protease interactions that modulate inflammatory processes can be important in sepsis. The most significant pathways by which coagulation factors regulate inflammation is by binding to protease-activated receptors (PARs). PARs are transmembrane G-protein coupled receptors and four different types (PAR 1-4) have been recognized. A typical property of PARs is that they serve as their own ligand. Proteolytic cleavage by an activated coagulation factor leads to exposure of a neoamino terminus, which is capable of activating the same receptor (and presumably adjacent receptors), leading to transmembrane signaling. PARs 1, 3, and 4 are receptors that are activated by thrombin while PAR-2 is triggered by the tissue factor-factor VIIa complex, factor Xa, and trypsin. PAR-1 is also a receptor for the tissue factor-factor VIIa complex and factor Xa.

### EXAMPLES

The disclosure is illustrated herein by the experiments described by the following examples, which should not be construed as limiting. Those skilled in the art will understand that this disclosure may be embodied in many different forms and should not be construed as limited to the aspects set forth herein. Rather, these examples are provided so that this disclosure will fully convey the disclosure to those skilled in the art. Many modifications and other aspects of the disclosure will come to mind in one skilled in the art to which this disclosure pertains having the benefit of the teachings presented in the foregoing description. Although specific terms are employed, they are used as in the art unless otherwise indicated.

### Example 1: Dosing of the Protease Inhibitor Component

The use of nafamostat in the antiviral cocktail for treatment of COVID-19 (and the potential use of other anticoagulants in the cocktail) requires a starting dose to ensure there is no interaction between the nafamostat and another medication utilized in the antiviral COVID treatment. Table 4 below outlines the starting dose and maintenance dose of nafamostat for subjects receiving no VTE prophylaxis, VTE prophylaxis specific to the agent and dose, and the dose of nafamostat for subjects receiving intermediate and full anticoagulation. Table 3 (above) outlines the various therapeutically active agents utilized for VTE prophylaxis and full dose anticoagulation at that corresponding level.

TABLE 4
Nafamostat Dosing

| | | VTE | | Subject already on |
|---|---|---|---|---|
| | | Prophylaxis at | Intermediate | Full Anticoagulant |
| | Absent of VTE | Standard | Anticoagulant | Dose that is not |
| Starting Dose | Prophylaxis Dose | Dose | Dose | from nafamostat |
| Nafamostat | 45-100 | 25-100 | 10-50 | 10-25 mcg/kg/hour |
| (cont. infusion) | mcg/kg/hour over | mcg/kg/hour | mcg/kg/hour | over 2 hours |
| | 2 hours | over 2 hours | over 2 hours | |
| Maintenance | 90-600 | 50-500 | 20-450 | 15-350 mcg/kg/hour |
| Dose | mcg/kg/hour for | mcg/kg/hour for | mcg/kg/hour for | for up to 21 days |
| (cont. infusion) | up to 21 days | up to 21 days | up to 21 days | |

### Example 2: Example Nafamostat Composition

Nafamostat can be infused as a sterile solution containing the following ingredients listed in Table 5. A reconstitution solvent such as water or saline solution may be used to dilute to the desired infusion concentration.

TABLE 5
Sample Product Vial Contents (100 mg vial)

| | Amount | |
|---|---|---|
| Component | (mg) | Purpose |
| Mannitol | 200 | Mannitol improves the appearance |
| | | and dissolution characteristics of |
| | | the lyophilized powder. |
| Nafamostat | 100 | Nafamostat is the therapeutically |
| | | active agent (API). |
| mesylate | | Succinic acid decreases the pH |
| | | of the solution, which |
| Succinic acid | 10 | improves the stability in water. |
| | | A lower pH solution is more |
| | | stable when dissolved with the |
| | | recommended diluent, 5% dextrose. |

Nafamostat may also be prepared as a non-aqueous solution in DMSO, which eliminates the need for lyophilization.

### Example 3: Use of Nafamostat as Ebola Virus Antiviral Agent

The following in vitro experiment is carried out to assess the antiviral activity of the nafamostat protease inhibitor component. Using tissue culture and pseudotype or recombinant virus transformed with and expressing the GP gene from the Ebola virus EBOV subtype (see, e.g., Chandran et al. (2005) Science 308:1643-1645; Feldmann et al. (2013) “Filoviridae: Marburg and Ebola viruses”, Chapter 32 in Fields Virology, 6^{th} ed., Knipe et al eds., pp 923-956; and Hunt et al. (2012) Viruses 4:258-275). The nafamostat composition of Example 2 is added to the tissue culture medium and the ability that composition to inhibit the proteolysis of the GP heterodimer and entry of the recombinant viral genome into the tissue culture cells is assessed.

Further studies can include in vivo animal models to assess the ability of the nafamostat composition of Example 2 to inhibit release of CatB (see, e.g., Chandran et al., supra and Marzi et al. (2012) Lancet 6:e1923).

Additional studies using in vivo animal models can also be carried out to assess the effect of the nafamostat composition of Example 2 on EBOV-induced DIC. Mice, guinea pigs and non-human primates are relevant models since EBOV infection induces thrombocytopenia in all three of these animals and non-human primates develop DIC (see, e.g., Bray et al. (2001) J. Comp. Pathol. 125:243-253).

### INCORPORATION BY REFERENCE

Each publication and patent mentioned herein is hereby incorporated by reference in its entirety. In case of conflict, the present application, including any definitions herein, will control.

### EQUIVALENTS

While specific aspects of the subject invention have been discussed, the above specification is illustrative and not restrictive. Many variations of the invention will become apparent to those skilled in the art upon review of this specification and the following claims. The full scope of the invention should be determined by reference to the claims, along with their full scope of equivalents, and the specification, along with such variations.

1. An antiviral cocktail composition comprising a therapeutically effective dose of a protease inhibitor component combined with a therapeutically effective dose of at least one additional component selected from: an anticoagulant agent; an anti-inflammatory agent; and an antiviral agent; wherein each said component is suitable for administration to a subject using a conjoined administration procedure.

2-5. (canceled)

6. The composition of claim 1, wherein the protease inhibitor component is provided as a dosage form that is suitable for parenteral administration.

7. The composition of claim 1, wherein the protease inhibitor component is selected from nafamostat, camostat and gabexate or any pharmaceutically acceptable salt thereof.

8-9. (canceled)

10. The composition of claim 1, further comprising a therapeutically effective dose of an antiviral agent.

11. (canceled)

12. The composition of claim 10, wherein the antiviral agent is provided as a dosage form that is suitable for parenteral administration.

13. The composition of claim 10, wherein the antiviral agent is provided as a dosage form that is suitable for oral administration.

14. The composition of claim 10, wherein the antiviral agent is selected from an interferon, a pegylated interferon, remdesivir (RDV), molnupiravir, nirmatrelvir, ritonavir, galidesivir, ASC09, danoprevir, AT-527, lopinavir, and combinations of ASC09/ritonavir, nirmatrelvir/ritonavir, lopinavir/ritonavir or danoprevir/ritonavir.

15. (canceled)

16. The composition of claim 1, further comprising a therapeutically effective dose of an anticoagulant agent.

17. (canceled)

18. The composition of claim 16, wherein the anticoagulant agent is provided as a dosage form that is suitable for parenteral administration.

19. The composition of claim 16, wherein the anticoagulant agent is selected from: heparin, enoxaparin, ardiparin, tinzaparin, dalteparin, fondaparinux, argatroban, apixaban, dabigatran, rivaroxaban, and edoxaban or any pharmaceutically acceptable salt thereof.

20. The composition of claim 1, further comprising a therapeutically effective dose of an anti-inflammatory agent.

21. (canceled)

22. The composition of claim 20, wherein the anti-inflammatory agent is provided as a dosage form that is suitable for parenteral administration.

23. The composition of claim 20, wherein the anti-inflammatory agent is selected from an adrenocortical steroid or an immunosuppressive.

24. A method of treating a viral infection in a subject comprising conjoint administration of the antiviral cocktail composition of claim 1 to the subject.

25. The method of claim 24, wherein a starting dose for the protease inhibitor component is from 5-200 mcg/kg/hour.

26-29. (canceled)

30. The method of claim 25, wherein the starting dose of the protease inhibitor component is administered to the subject for at least about 2 hours.

31. The method of claim 24, wherein a maintenance dose for the protease inhibitor component is from 10-700 mcg/kg/hour.

32-35. (canceled)

36. The method of claim 31, wherein the maintenance dose of the protease inhibitor component is administered to the subject for up to about 21 days.

37. The method of claim 24, wherein the subject is treated for viral infection from SARS-CoV-2, influenza, Ebola virus, Dengue, West Nile, or a respiratory syncytial virus.

38. The method of claim 24, wherein the viral infection is characterized by hypoxemia, pulmonary intravascular coagulopathy, disseminated intravascular coagulation, viremia, septic shock, cytokine storm, systemic inflammatory response syndrome (SIRS), acute respiratory distress syndrome (ARDS), and/or vascular leak syndrome in the subject.
